SEQUENCE LISTING (10 sequences)

SEQ ID NO: 1 - DNA, 1710 bp, Solanum tuberosum
  source 1..1710: mol_type = genomic DNA
atgggaaggtctccttgttgtgatgaaaatgggctgaaaaaaggaccttggactcctgaagaagatcaaatacttgttgattatattgacaaacatggtcatggaagttggagagcccttcctaagcttgcaggtattataataattccaacaaaaaaataataattttttttatcactactaaaaatggaattagctacgagcttcgtcgctggtccatctatggtttacttgtaactaatgaaaattcgttgctaagtgggattagcaacaacctctttttggcatgagcaaaggtagctagctaggggcgcgtaagggatttatctgaatattttttttcttcagaaattatattatatagagagagttaaaattatatatggtttatgtatatataatagatgttgaattttctttgcttctgatttcgataggtaagttcttagttataacatagatgatatttgttttaacaaaactaacatttgttgtttttatatgctatataggacttaataggtgtgggaagagctgcaggttaagatggactaattatttgaggcctgatatcaagagaggcaaattttctgaagaagaagaacaaacaattcttcatctccattccatcctaggaaacaagtaattaattctttcaatataacttacattatttataggtcagtaaaaaatatagagaactttggtatatttttaatcgtattttatataatattgactaaataataagcttaaataatgtggggttactaatggataatggtgatttatatagttgacctcaattatgctagagatttatgcacaattattgttgttgttgctaacacatttgttttcttgtttgtgaaaattcatattgatgatatcttttgatattatggttgtagatggtccgcgatcgcaacacatttatcaggaagaacagacaatgagatcaagaatttctggaacacacatttaaagaagaagctaattcaaatgggatatgatccaatgacacacaagccaagaactgatttatttgcaaacttgcctaatataatagctttggcaaatttacttcaacatcatccactagaagatcatgctgtgagattacaagtacaagctgcccaattagctaaaattcaatatttacaatttctattccaatcttcaaataattataatccaacacaacctacaccctcatcaaacactcaacattacaataataataataatattaatcttggagatttaggggcatttaatttgacaaattttgttaaagaaaatagtaccccttctttaaatttatccaatattgaaaatcaaacacttttctccaatgaaaataactcttgctctcaactactccacaataatcaagataatacacaacaagtccctttcaatttccaaacacatttgaataataatattaatgaaattagtgacaatattaattgtcacaattttaattttgatcaagaaaataattcaactagttccccattaaatattactaatattcttccttctccacattcttcaagtcatttaccacctttggctgaaatttcaattagcaataatcaaggagattctagcagtaattcaactaacaatggtgctgatcaagggagttcttcatattggccagaattattttttgaagaacacttcatgcatgacatttcttga

SEQ ID NO: 2 - DNA, 1817 bp, synthetic construct
  source 1..1817: mol_type = other DNA
cgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgggaagagctgcaggttaagagttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatattaatccaaactactgcagcctgacagacaaatgaggatgcaaacaattttaaagtttatctaacgctagctgttttgtttcttctctctggtgcaccaacgacggcgttttctcaatcataaagaggcttgttttacttaaggccaataatgttgatggatcgaaagaagagggcttttaataaacgagcccgtttaagctgtaaacgatgtcaaaaacatcccacatcgttcagttgaaaatagaagctctgtttatatattggtagagtcgactaagagattgctaacaatggtgctgatcaagttttagagctagaaatagcaagttaaaataaggctagtccgttatcaacttgaaaaagtggcaccgagtcggtgctttttttggatagaatttcccagcttttttgcgtgtttcagctctcatgatccttggccaatgggtgtagtaaattttctgcacattcattggatggaaaataatggttttagctttagggaataagaaaagtgtataggaaggggatttttgtacaatcacatttgaattaggtctttgaaatgacagggaatgaggacatatgatgagacggtcattgttttagttccaccacgattatatttgaaatttacgtgagtgtgagtgagacttgcataagaaaataaaatctttagttgggaaaaaattcaataatataaatgggcttgagaaggaagcgagggataggcctttttctaaaataggcccatttaagctattaacaatcttcaaaagtaccacagcgcttaggtaaagaaagcagctgagtttatatatggttagagacgaagtagtgattgtgacttgaagaatgtggagagttttagagctagaaatagcaagttaaaataaggctagtccgttatcaacttgaaaaagtggcaccgagtcggtgcttttttttggcaaaaattttcagattttttcttcatctgtagatttctgggtttttttttccgtttcgtgaatcataagtgaagttttggatgcaaatctgcgcgaaaaaagttggacctgcaatgagcttatttagatagctaagacaaagtgattggtccgtt

SEQ ID NO: 3 - DNA, 20 bp, synthetic construct
  source 1..20: mol_type = other DNA
cacgcaggttctccggccgc

SEQ ID NO: 4 - DNA, 20 bp, synthetic construct
  source 1..20: mol_type = other DNA
tgcgctgcgaatcgggagcg

SEQ ID NO: 5 - DNA, 21 bp, synthetic construct
  source 1..21: mol_type = other DNA
catgtctaatttggtagcatc

SEQ ID NO: 6 - DNA, 20 bp, synthetic construct
  source 1..20: mol_type = other DNA
gaaatgtcatgcatgaagtg

SEQ ID NO: 7 - DNA, 23 bp, synthetic construct
  source 1..23: mol_type = other DNA
ctagcagcatgaagattaaggtg

SEQ ID NO: 8 - DNA, 23 bp, synthetic construct
  source 1..23: mol_type = other DNA
ggacaatagaaggaccagattcg

SEQ ID NO: 9 - DNA, 21 bp, synthetic construct
  source 1..21: mol_type = other DNA
attggaaacggatatgctcca

SEQ ID NO: 10 - DNA, 21 bp, synthetic construct
  source 1..21: mol_type = other DNA
tccttacctgaacgcctgtca

(19) United States
(12) Patent Application Publication
(10) Pub. No.: US 2024/0423155 A1
(43) Pub. Date: Dec. 26, 2024

(54) POTATO VARIETY NAMED 'UNR-01'

(71) Applicant: Board of Regents of the Nevada System of Higher Education, on Behalf of the University of Nevada, Reno, NV (US)

(72) Inventors: Vijaya Kumar Reddy Vulavala, Ridge, NY (US); Dylan K. Kosma, Reno, NV (US); Patricia Santos, Reno, NV (US)

(73) Assignee: Board of Regents of the Nevada System of Higher Education, on Behalf of the University of Nevada, Reno, NV (US)

(21) Appl. No.: 18/739,142

(22) Filed: Jun. 10, 2024

Related U.S. Application Data
(60) Provisional application No. 63/509,898, filed on Jun. 23, 2023.

(51) Int. Cl. A01H 6/82; A01H 5/06

(52) U.S. Cl. CPC A01H 6/827; A01H 5/06

(57) ABSTRACT

Herein provided is a new potato variety designated ‘UNR-01’ as well as the tubers, seeds, plants, plant parts, and derivatives of the new potato variety ‘UNR-01’. This transgenic variety was generated using CRISPR-Cas9 and deleting a portion of the StMYB93 (PGSC0003DMG400006408) gene. This transgenic variety has a sprout suppressed phenotype that results in a longer storage life in cold storage conditions without the application of sprout-inhibiting chemicals. These potatoes were not impacted in reduced sugar levels, wound healing capacities, and native periderm suberin content. Also provided are tissue cultures of the new potato variety ‘UNR-01’ and the plants regenerated therefrom. Methods for producing potato plants by crossing the new potato variety ‘UNR-01’ with itself or another potato variety and plants produced by such methods are also provided. ‘UNR-01’ potatoes exhibit suppressed sprouting and longer storage life in cold storage conditions without the application of sprout-inhibiting chemicals.

### CROSS-REFERENCE TO RELATED APPLICATION

This application claims priority to U.S. Provisional Application No. 63/509,898 filed Jun. 23, 2023, which is herein incorporated by reference in its entirety.

### ACKNOWLEDGMENT OF GOVERNMENT SUPPORT

This invention was made with government support under 1547713 awarded by the National Science Foundation. The government has certain rights in the invention.

### FIELD

This disclosure provides a new and distinctive potato variety ‘UNR-01’ and to the tubers, plants, plant parts, tissue culture, and seeds of potato variety ‘UNR-01’. Also provided are food products produced from potato variety ‘UNR-01’, such as French fries, potato chips, dehydrated potato material, potato flakes, and potato granules.

### INCORPORATION OF SEQUENCE LISTING

The Sequence Listing is submitted as an XML file in the form of the file named “Sequence.xml” (13, 151 bytes), which was created on Jun. 3, 2024 which is incorporated by reference herein.

### BACKGROUND

Potato is fourth most-consumed food crop in the world and the number one vegetable crop in the USA with a gross production of ˜42 billion pounds/year, and annual crop value of $4.14 billion (FAO stats, 2019), $3.9 billion for 2020 and $4.06 billion for 2021 (nationalpotatocouncil.org). In addition to carbohydrates, potatoes are enriched with vitamin C, potassium, proteins and dietary fiber. To sustain a year-round supply of potatoes, tubers are stored for extended periods of time and, where possible, under climate-controlled conditions (Sonnewald, 2001). Roughly 35% of the annual potato tuber crop is lost each year under postharvest storage conditions equating to approximately $1.7 billion of lost potential crop value, due to poor storage life (N. A. S. S. USDA, “Potatoes 2013 summary,” September 2014). Premature sprouting and rots are major culprits behind these losses. Sprouting of potatoes is associated with several factors such as tuber dormancy, photoperiodicity, temperature differences and humidity. A tuber is considered dormant (endodormancy) when it has not sprouted for a period of time following its storage. Tuber sprouting during storage has adverse effects on tuber quality such as increased water loss, higher levels of reduced sugars which lead to browning during the frying process, and higher levels of glycoalkaloids such as α-solanine and α-chaconine, rendering the tubers unsuitable for processing and consumption. Furthermore, a recent ban on major chemical sprout suppressors such as CIPC [isopropyl N-(3-chlorophenyl) carbamate] has created a greater demand for alternative approaches to sprout suppression (e.g., Sonnewald and Sonnewald, Planta, 239:27-38, 2014; Blenkinsop et al., J. Agricultural and Food Chem., 50 (16): 4545-4553, 2002; Camps and Camps, Molecules, 24 (5): 967, 2019).

Tuber rots (microbial pathogens) are other major culprits for potato tuber post-harvest storage losses. Harvesting and post-harvest storage practices results in damage to tubers (e.g., bruising and wounding). Tuber wound sites provide a convenient entry point for microbial pathogens and poor wound healing capacity is associated with reduced resistance to tuber rots. Potatoes heal wounds by rapidly forming a closing layer comprised of polymerized lipids and phenolics followed by production of new cells in the wound periderm that are heavily enriched with a lipid-phenolic biopolymer termed suberin. Wound healing capacity is determined by several factors such as cultivar, tuber age, abscisic acid content and storage conditions such as temperature and humidity. Thus, there is a need for potatoes with the ability to be stored longer by suppressing sprouting.

### SUMMARY

Provided herein is a new potato variety generated using CRISPR-Cas9 gene-editing of StMYB93 (PGSC0003DMG400006408), a transcription factor (TF) belonging to the Myeloblastosis (MYB) gene family. This gene is a potential regulator of wound suberin. One transgenic event, Stmyb93-20 (UNR-01′), displayed unexpected and beneficial phenotypes during storage including sprout suppression and a greater number of tubers when grown in soil. Tubers of this new variety ‘UNR-01’ did not differ from non-gene-edited wildtype for traits important for potato chip production and storage including reduced sugar levels and wound healing, respectively. Furthermore, tubers from ‘UNR-01’ maintained a smooth skin with minimal tuber shrinkage during long-term storage.

A deposit of the new potato variety ‘UNR-01’ will be made to meet all of the requirements of 37 C.F.R. §§ 1.801-1.809. The deposit will be maintained in the depository for a period of 30 years, or 5 years after the last request, or for the effective life of the patent, whichever is longer, and will be replaced if necessary, during that period. In one embodiment, the disclosure provides a potato tuber, plantlet, or tissue culture of potato variety ‘UNR-01’ deposited under Accession No. ___.

The disclosure provides potato plants including or consisting of all of the morphological and physiological characteristics of ‘UNR-01’, such as the characteristics shown in FIGS. 1-6 and 8, for example, reduced sprouting suppression phenotype, broken apical dominance resulting in the formation of multiple branches, larger number of tubers smaller in size, and a genetic deletion of about 1.2 kB in an StMYB93 gene. In some examples, the morphological and physiological characteristics of ‘UNR-01’ include similar sugar levels and wound healing to that of potatoes cv. Atlantic. Also provided are seeds of such plants, progeny of such plants, and parts of such plants (such as pollen, ovules, and cells). In one example, the disclosure provides potato plants having the genotype of ‘UNR-01’. For example, the disclosure provides plants produced by growing the seed, tubers, or tissue cultures of the new potato variety ‘UNR-01’.

The disclosure provides a tissue culture of regenerable cells of the new potato variety ‘UNR-01’, as well as plants regenerated therefrom. Such regenerated potato plants can include or consist of all of the physiological and morphological characteristics of a plant grown from the seed, tissue culture, or tuber of the new potato variety ‘UNR-01’. Exemplary regenerable cells include but are not limited to those from protoplasts or cells, such as those from embryos, meristematic cells, pollen, leaves, roots, root tips, anthers, pistils, flowers, seed, cotyledons, hypocotyls, shoots, ovule, light sprout, petiole, eye, stem, tuber, or stems of the new potato variety ‘UNR-01’.

Also provided are compositions that include ‘UNR-01’ cells, plants, or tubers comprised in plant seed growth media, such as a soil or a synthetic cultivation medium.

The disclosed ‘UNR-01’ plants, seeds, and cells can further include a transgene, such as a transgene introduced by backcrossing or genetic transformation into potato variety ‘UNR-01’. In some examples, the transgene confers one or more of herbicide tolerance (or resistance), resistance to a bacterial disease, resistance to a viral disease, resistance to an insect, resistance to a fungal disease, resistance to a nematode, resistance to a pest, male sterility, site-specific recombination, abiotic stress tolerance, modified linolenic or linoleic acid content, modified antioxidant characteristics, modified essential seed amino acid characteristics, modified fatty acid metabolism, modified carbohydrate metabolism, modified phytic acid metabolism, modified protein metabolism, water stress resistance, restoration of male fertility, altered starch, thermotolerant amylase, or other improved nutritional qualities. In some examples, the disclosed ‘UNR-01’ plants and cells further include a single transgene, and include all of the physiological and morphological characteristics of ‘UNR-01’ (for example in addition to the new trait(s) conferred by the transgene).

The disclosed ‘UNR-01’ plants, tubers, seeds, and cells can further include a single locus conversion. For example, provided is a potato plant that includes a single locus conversion introduced by backcrossing or genetic transformation into potato variety ‘UNR-01’. Also provided is potato seed that includes a single locus conversion introduced by backcrossing or genetic transformation into potato variety ‘UNR-01’, and obtaining seed therefrom. In some examples, a single locus conversion confers one or more of herbicide tolerance (or resistance), resistance to a bacterial disease, resistance to a viral disease, resistance to an insect, resistance to a fungal disease, resistance to a nematode, resistance to a pest, male sterility, site-specific recombination, abiotic stress tolerance, modified linolenic or linoleic acid content, modified antioxidant characteristics, modified essential seed amino acid characteristics, modified fatty acid metabolism, modified carbohydrate metabolism, modified phytic acid metabolism, modified protein metabolism, water stress resistance, restoration of male fertility, altered starch, thermotolerant amylase, or other improved nutritional qualities. In some examples, the disclosed ‘UNR-01’ plants and cells further include a single locus conversion, and can include all of the physiological and morphological characteristics of ‘UNR-01’ (for example in addition to the new trait(s) conferred by the single locus conversion).

Methods of producing seed potatoes (having at least one eye) from the ‘UNR-01’ potato plants are provided. In some examples, such methods include crossing ‘UNR-01’ with itself or a second potato plant and harvesting a resulting potato and generating seed potatoes therefrom. In a cross, either parent may serve as the male or female. In some examples, the second potato plant has one or more desirable traits, which is/are introduced into (e.g., via transformation) plants and seeds resulting from such a cross. For example, the second plant can be transgenic, wherein the transgene confers the desirable trait(s). Seed potatoes produced by such methods, including F₁ hybrid seed potatoes, as well as potato plants or parts thereof (including F₁ plants) produced by growing such a seed potato, are provided. In some examples, the method of crossing includes planting seed potatoes of the new potato variety ‘UNR-01’, cultivating potatoes resulting from the seed potatoes, and generating seed potatoes from the cultivated potatoes.

Methods are provided for producing a plant derived from potato variety ‘UNR-01’, which has one or more added traits, as well as plants, tubers, seed potatoes, and cells generated from such methods. In one example, such a method provides a potato plant having a single locus conversion of the new potato variety ‘UNR-01’, wherein the potato plant in some examples includes or expresses all of the physiological and morphological characteristics of the new potato variety ‘UNR-01’ (such as those shown in FIGS. 1-6 and 8). In some embodiments, the single locus conversion can include a dominant or recessive allele. Such methods can include introducing a transgene that confers one or more additional traits into a plant of the new potato variety ‘UNR-01’ (e.g., via transformation). Exemplary additional traits include herbicide tolerance (or resistance), resistance to a bacterial disease, resistance to a viral disease, resistance to an insect, resistance to a fungal disease, resistance to a nematode, resistance to a pest, male sterility, site-specific recombination, abiotic stress tolerance, modified linolenic or linoleic acid content, modified antioxidant characteristics, modified essential seed amino acid characteristics, modified fatty acid metabolism, modified carbohydrate metabolism, modified phytic acid metabolism, modified protein metabolism, water stress resistance, restoration of male fertility, altered starch, thermotolerant amylase or other improved nutritional qualities.

Methods of introducing a single locus conversion (such as an additional trait) into the new potato variety ‘UNR-01’ are provided. In some examples, the methods include (a) crossing a plant of variety ‘UNR-01’ with a second plant having one or more additional traits to produce F₁ progeny plants; (b) selecting F₁ progeny plants that have the additional trait to produce selected F₁ progeny plants; (c) crossing the selected progeny plants with at least a first plant of variety ‘UNR-01’ to produce backcross progeny plants; (d) selecting backcross progeny plants that have the additional trait and in some examples some or all of the physiological and morphological characteristics of potato variety ‘UNR-01’ to produce selected backcross progeny plants; and (e) repeating steps (c) and (d) one or more times in succession to produce selected second or higher backcross progeny plants that include the additional trait and in some examples some or all of the physiological and morphological characteristics of potato variety ‘UNR-01’ when grown in the same environmental conditions. In some embodiments, the single locus confers a desirable trait, such as herbicide tolerance (or resistance), resistance to a bacterial disease, resistance to a viral disease, resistance to an insect, resistance to a fungal disease, resistance to a nematode, resistance to a pest, male sterility, site-specific recombination, abiotic stress tolerance, modified linolenic or linoleic acid content, modified antioxidant characteristics, modified essential seed amino acid characteristics, modified fatty acid metabolism, modified carbohydrate metabolism, modified phytic acid metabolism, modified protein metabolism, water stress resistance, restoration of male fertility, altered starch, thermotolerant amylase or other improved nutritional qualities. In some examples, the single locus confers the ability to synthesize a protein encoded by a gene located within the single locus.

Methods of producing a potato plant derived from the new potato variety ‘UNR-01’, such as an inbred potato plant, are provided. In particular examples the method includes (a) preparing a progeny plant derived from the new potato variety ‘UNR-01’ by crossing a plant of ‘UNR-01’ with a potato plant of a second variety; and (b) crossing the progeny plant with itself or a second plant to produce a progeny plant of a subsequent generation which is derived from a plant of the new potato variety ‘UNR-01’. In some embodiments, the method further includes (c) growing a progeny plant of a subsequent generation from seed potatoes and crossing the progeny plant of a subsequent generation with itself or a second plant; and (d) repeating steps (b) and (c) for at least 2 additional generations (such as at least 3, at least 5, or at least 10 additional generations, such as 2, 3, 4, 5, 6, 7, 8, 9, 10, 11, 12, 13, 14, 15, 16, 17, 18, 19 or 20 additional generations) with sufficient inbreeding to produce an inbred potato plant derived from ‘UNR-01’. In other examples, the method includes (a) crossing a potato plant derived from the new potato variety ‘UNR-01’ with itself or another potato plant to yield additional potato variety ‘UNR-01’-derived progeny seed potatoes; (b) growing the progeny seed potatoes of (a) under plant growth conditions, to yield additional potato variety ‘UNR-01’-derived potato plants; and (c) repeating the crossing and growing steps of (a) and (b) from 0 to 7 times (such as 0 to 4 or 1 to 5 times, such as 0, 1, 2, 3, 4, 5, 6, or 7 times) to generate further potato variety ‘UNR-01’-derived potato plants.

Methods are provided for developing a new potato plant using ‘UNR-01’. For example, the methods can include using ‘UNR-01’ plants or parts thereof as a source of breeding material in plant breeding techniques, such as recurrent selection, mass selection, bulk selection, backcrossing, pedigree breeding, genetic marker-assisted selection, selfing, outcrossing, haploid production, doubled haploid production, and genetic transformation. In some examples, a plant of the new potato variety ‘UNR-01’ is used as the male or female parent. Such a method can further include (a) crossing a progeny potato plant derived from ‘UNR-01’ with itself or a second plant to produce a seed of a progeny plant of a subsequent generation; (b) growing the progeny plant of the subsequent generation from seed potatoes of the progeny plant of the subsequent generation; and (c) repeating steps (a) and (b) for at least one additional generation (such as 3-10 generations) to produce a progeny potato plant further derived from the potato variety ‘UNR-01’.

The disclosure provides a first generation (F₁) hybrid potato, plants, and parts thereof, produced by crossing a plant of the new potato variety ‘UNR-01’ to a second potato plant. In some embodiments, the F₁ hybrid potato is grown from a hybrid seed potato produced by crossing the new potato variety ‘UNR-01’ to a second potato plant. In specific examples, provided is a seed potato of an F₁ hybrid plant produced with the new potato variety ‘UNR-01’ as one parent, the second generation (F₂) hybrid potato plant grown from the seed potato of the F₁ hybrid plant, and the seed potato of the F₂ hybrid plant.

Methods of producing hybrid potatoes are also provided. In one example the method includes crossing the new potato variety ‘UNR-01’ to a second, distinct potato plant which is nonisogenic to the new potato variety ‘UNR-01’. In some examples, the method includes cultivating ‘UNR-01’ potato plants and cultivating potato plants grown from a second, distinct potato plant, until the plants bear flowers. A flower on one of the two plants is cross pollinated with the pollen of the other plant, and the potatoes resulting from such a cross are harvested.

The disclosure also provides potato plants and parts thereof produced by any of the methods disclosed herein. Thus, provided herein are plants of potato variety ‘UNR-01’ that further include a single locus conversion, such as one or more additional traits, for example produced by backcrossing or genetic transformation. In some embodiments, the potato plants produced by the disclosed methods include essentially all, or all of the traits of the new potato variety ‘UNR-01’ as described herein.

Methods are also provided for producing a genetic marker profile, which can include extracting nucleic acids from ‘UNR-01’ seed potato or a plant grown from such seed potato, and genotyping said nucleic acids, thereby producing a genetic marker profile.

Methods of plant breeding are also provided. In one example, such a method includes isolating nucleic acids from a cell from ‘UNR-01’, identifying one or more polymorphisms from the isolated nucleic acids, and selecting a plant having one or more polymorphisms, wherein the plant is used in a plant breeding method.

Also provided are methods of producing nucleic acids, wherein the method can include extracting nucleic acids from an F₁ potato plant or part thereof produced by potato plant ‘UNR-01’.

Methods of producing a commodity plant product are provided. In some examples the method includes obtaining or supplying a plant of the new potato variety ‘UNR-01’, or a part thereof, and producing the commodity plant product therefrom. In some examples the method includes growing and harvesting the plant, or a part thereof. Exemplary commodity plant products include but are not limited to French fries, potato chips, dehydrated potato material, potato flakes, and potato granules.

The foregoing and other objects and features of the disclosure will become more apparent from the following detailed description which proceeds with reference to the accompanying figures.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1C show the dormant and sprouting phenotypes observed in StMYB93 gene-edited tubers (‘UNR-01’) and in wild type (WT) Atlantic tubers. ‘UNR-01’ demonstrated prolonged tuber dormancy, and less shrinkage compared with WT tubers. All tubers were stored for ˜10 months at 12° C. (A) WT tubers demonstrated longer sprout growth whereas ‘UNR-01’ demonstrated reduced sprouting and less shrinkage with broken apical dominance. (B) Closer view of WT tuber sprouts. (C) Closer observation of ‘UNR-01’ demonstrating inhibited sprouting, reduced sprout length, smooth skins, reduced shrinkage (less water loss), and broken apical dominance.

FIG. 2 Digital image showing branching phenotype of ‘UNR-01’ shoots. Loss of apical dominance observed in sprouting ‘UNR-01’ was also observed in above-ground vegetative growth. (WT) Atlantic plants generated thick single stems from each “eye” whereas ‘UNR-01’ tubers produced multiple shoots from a single “eye”. The arrow in the far-right picture highlights the extensive branching observed from sprouts derived from an individual “eye.

FIG. 3 shows ‘UNR-01’ tuber yields and sizes. ‘UNR-01’ presented increased numbers of tubers per plant but with equivalent total per plant tuber mass yields. Average single tuber weights from ‘UNR-01’ plants were smaller. Above ground canopy dry weights were equal.

FIG. 4 shows measurements of reduced sugar levels (glucose). The color change on the test strips shows the same amounts of reduced sugars (glucose) in wildtype and ‘UNR-01’. A color scale on top right corner demonstrates the percentage of reduced sugars (glucose). Kit details can be found on the internet on the bartovation website.

FIGS. 5A-5B show wound suberin amounts and composition of native periderm (A), and 7 days post wounding periderms (B) from wildtype (WT) and ‘UNR-01’ (GE-Stmyb93-20) tubers. The total suberin amounts in Native periderm (skin) and wound periderm (wounded sites) in gene-edited Stmyb93-20 mutant ‘UNR-01 were not changed. Native periderm suberin composition showed a minor reduction in only 2 suberin components in the gene-edited Stmyb93-20 mutant ‘UNR-01’.

FIG. 6 is a series of bar graphs showing relative expression levels of known suberin genes demonstrating unchanged expression levels of two suberin genes at 7 days post wounding (7 dpw) in ‘UNR-01’ compared to wild type. One gene, StCYP86A33 showed a minor reduction in expression level. The relative expression levels were analyzed by normalizing with 2 house-keeping genes StActin and StEF1-alpha genes. Asterisks indicates statistically significant differences.

FIG. 7 is a plasmid map showing the individual elements of pDIRECT_22A along with the cloned gRNA cassette in it.

FIG. 8 is a digital image of an agarose gel showing PCR amplicons from genomic DNA of wild type and individual edited events of stmyb93. The genomic DNA from all individual plants was extracted and PCR amplified a region of StMYB93, making sure that the targeted gRNAs portions were included, and loaded on the gel. Wild type showed a single large band, whereas the stmyb93edited events showed different size of bands demonstrating the allele specific editing patterns in the gel. ‘UNR-01’ is Stmyb93-20.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NO: 1 is an exemplary genomic DNA sequence of StMYB93
(PGSC0003DMG400006408) demonstrating the gRNA targets (underlined) and PAM
region (bold)
ATGGGAAGGTCTCCTTGTTGTGATGAAAATGGGCTGAAAAAAGGACCTTGGACTCCT
GAAGAAGATCAAATACTTGTTGATTATATTGACAAACATGGTCATGGAAGTTGGAG
AGCCCTTCCTAAGCTTGCAGGTATTATAATAATTCCAACAAAAAAATAATAATTTTT
TTTATCACTACTAAAAATGGAATTAGCTACGAGCTTCGTCGCTGGTCCATCTATGGTT
TACTTGTAACTAATGAAAATTCGTTGCTAAGTGGGATTAGCAACAACCTCTTTTTGG
CATGAGCAAAGGTAGCTAGCTAGGGGCGCGTAAGGGATTTATCTGAATATTTTTTTT
CTTCAGAAATTATATTATATAGAGAGAGTTAAAATTATATATGGTTTATGTATATAT
AATAGATGTTGAATTTTCTTTGCTTCTGATTTCGATAGGTAAGTTCTTAGTTATAACA
TAGATGATATTTGTTTTAACAAAACTAACATTTGTTGTTTTTATATGCTATATAGGAC
TTAATAGGTGTGGGAAGAGCTGCAGGTTAAGATGGACTAATTATTTGAGGCCTGAT
ATCAAGAGAGGCAAATTTTCTGAAGAAGAAGAACAAACAATTCTTCATCTCCATTCC
ATCCTAGGAAACAAGTAATTAATTCTTTCAATATAACTTACATTATTTATAGGTCAGT
AAAAAATATAGAGAACTTTGGTATATTTTTAATCGTATTTTATATAATATTGACTAA
ATAATAAGCTTAAATAATGTGGGGTTACTAATGGATAATGGTGATTTATATAGTTGA
CCTCAATTATGCTAGAGATTTATGCACAATTATTGTTGTTGTTGCTAACACATTTGTT
TTCTTGTTTGTGAAAATTCATATTGATGATATCTTTTGATATTATGGTTGTAGATGGT
CCGCGATCGCAACACATTTATCAGGAAGAACAGACAATGAGATCAAGAATTTCTGG
AACACACATTTAAAGAAGAAGCTAATTCAAATGGGATATGATCCAATGACACACAA
GCCAAGAACTGATTTATTTGCAAACTTGCCTAATATAATAGCTTTGGCAAATTTACTT
CAACATCATCCACTAGAAGATCATGCTGTGAGATTACAAGTACAAGCTGCCCAATTA
GCTAAAATTCAATATTTACAATTTCTATTCCAATCTTCAAATAATTATAATCCAACAC
AACCTACACCCTCATCAAACACTCAACATTACAATAATAATAATAATATTAATCTTG
GAGATTTAGGGGCATTTAATTTGACAAATTTTGTTAAAGAAAATAGTACCCCTTCTT
TAAATTTATCCAATATTGAAAATCAAACACTTTTCTCCAATGAAAATAACTCTTGCTC
TCAACTACTCCACAATAATCAAGATAATACACAACAAGTCCCTTTCAATTTCCAAAC
ACATTTGAATAATAATATTAATGAAATTAGTGACAATATTAATTGTCACAATTTTAA
TTTTGATCAAGAAAATAATTCAACTAGTTCCCCATTAAATATTACTAATATTCTTCCT
TCTCCACATTCTTCAAGTCATTTACCACCTTTGGCTGAAATTTCAATTAGCAATAATC
AAGGAGATTCTAGCAGTAATTCAACTAACAATGGTGCTGATCAAGGGAGTTCTTCA
TATTGGCCAGAATTATTTTTTGAAGAACACTTCATGCATGACATTTCTTGA
SEQ ID NO: 2 a gRNA cassette including individual elements and sequences
-(U6-26p)-(Target-1)-(gRNA-Sc)-(U6-26t)-(U6-29p)-(Target-2)-(gRNA-Sc)-
(U6-26t)-(U6-1p)- (Target-3)-(gRNA-Sc)- (U6-1t)-
CGACTTGCCTTCCGCACAATACATCATTTCTTCTTAGCTTTTTTTCTTCTTCTTCGTTC
ATACAGTTTTTTTTTGTTTATCAGCTTACATTTTCTTGAACCGTAGCTTTCGTTTTCTT
CTTTTTAACTTTCCATTCGGAGTTTTTGTATCTTGTTTCATAGTTTGTCCCAGGATTAG
AATGATTAGGCATCGAACCTTCAAGAATTTGATTGAATAAAACATCTTCATTCTTAA
GATATGAAGATAATCTTCAAAAGGCCCCTGGGAATCTGAAAGAAGAGAAGCAGGCC
CATTTATATGGGAAAGAACAATAGTATTTCTTATATAGGCCCATTTAAGTTGAAAAC
AATCTTCAAAAGTCCCACATCGCTTAGATAAGAAAACGAAGCTGAGTTTATATACAG
CTAGAGTCGAAGTAGTGATTGGGAAGAGCTGCAGGTTAAGAGTTTTAGAGCTAGAA
ATAGCAAGTTAAAATAAGGCTAGTCCGTTATCAACTTGAAAAAGTGGCACCGAGTC
GGTGCTTTTTTTTGCAAAATTTTCCAGATCGATTTCTTCTTCCTCTGTTCTTCGGCGTT
CAATTTCTGGGGTTTTCTCTTCGTTTTCTGTAACTGAAACCTAAAATTTGACCTAAAA
AAAATCTCAAATAATATGATTCAGTGGTTTTGTACTTTTCAGTTAGTTGAGTTTTGCA
GTTCCGATGAGATAAACCAATATTAATCCAAACTACTGCAGCCTGACAGACAAATG
AGGATGCAAACAATTTTAAAGTTTATCTAACGCTAGCTGTTTTGTTTCTTCTCTCTGG
TGCACCAACGACGGCGTTTTCTCAATCATAAAGAGGCTTGTTTTACTTAAGGCCAAT
AATGTTGATGGATCGAAAGAAGAGGGCTTTTAATAAACGAGCCCGTTTAAGCTGTA
AACGATGTCAAAAACATCCCACATCGTTCAGTTGAAAATAGAAGCTCTGTTTATATA
TTGGTAGAGTCGACTAAGAGATTGCTAACAATGGTGCTGATCAAGTTTTAGAGCTAG
AAATAGCAAGTTAAAATAAGGCTAGTCCGTTATCAACTTGAAAAAGTGGCACCGAG
TCGGTGCTTTTTTTGGATAGAATTTCCCAGCTTTTTTGCGTGTTTCAGCTCTCATGATC
CTTGGCCAATGGGTGTAGTAAATTTTCTGCACATTCATTGGATGGAAAATAATGGTT
TTAGCTTTAGGGAATAAGAAAAGTGTATAGGAAGGGGATTTTTGTACAATCACATTT
GAATTAGGTCTTTGAAATGACAGGGAATGAGGACATATGATGAGACGGTCATTGTTTTA
GTTCCACCACGATTATATTTGAAATTTACGTGAGTGTGAGTGAGACTTGCATAAG
AAAATAAAATCTTTAGTTGGGAAAAAATTCAATAATATAAATGGGCTTGAGAAGGA
AGCGAGGGATAGGCCTTTTTCTAAAATAGGCCCATTTAAGCTATTAACAATCTTCAA
AAGTACCACAGCGCTTAGGTAAAGAAAGCAGCTGAGTTTATATATGGTTAGAGACG
AAGTAGTGATTGTGACTTGAAGAATGTGGAGAGTTTTAGAGCTAGAAATAGCAAGT
TAAAATAAGGCTAGTCCGTTATCAACTTGAAAAAGTGGCACCGAGTCGGTGCTTTTT
TTTGGCAAAAATTTTCAGATTTTTTCTTCATCTGTAGATTTCTGGGTTTTTTTTTCCGT
TTCGTGAATCATAAGTGAAGTTTTGGATGCAAATCTGCGCGAAAAAAGTTGGACCTG
CAATGAGCTTATTTAGATAGCTAAGACAAAGTGATTGGTCCGTT

### DETAILED DESCRIPTION

### Description of Terms

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. For example, reference to “comprising a plant” includes one or a plurality of such plants. The term “or” refers to a single element of stated alternative elements or a combination of two or more elements, unless the context clearly indicates otherwise. For example, the phrase “A or B” refers to A, B, or a combination of both A and B. Furthermore, the various elements, features and steps discussed herein, as well as other known equivalents for each such element, feature or step, can be mixed and matched by one of ordinary skill in this art to perform methods in accordance with principles described herein. Among the various elements, features, and steps, some will be specifically included and others specifically excluded in particular examples.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and are not intended to be limiting.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−20% variation of the value it describes. Accordingly, in some embodiments, the numerical parameters set forth herein are approximations that can vary depending upon the desired properties sought to be obtained by a particular embodiment.

Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

Asexual propagation/reproduction: Producing progeny by generating an entire plant from leaf cuttings, stem cuttings, root cuttings, tuber eyes, stolons, single plant cells protoplasts, callus and the like, that does not involve fusion of gametes.

Backcross: The mating of a hybrid to one of its parents. For example hybrid progeny, for example a first generation hybrid (F₁), can be crossed back one or more times to one of its parents. Backcrossing can be used to introduce one or more single locus conversions (such as one or more desirable traits) from one genetic background into another.

Cell: Cell as used herein includes a plant cell, whether isolated, in tissue culture or incorporated in a plant or plant part.

Cotyledon: A type of seed leaf containing the food storage tissues of the seed.

Cross: Synonymous with hybridize or crossbreed. Includes the mating of genetically different individual plants, such as the mating of two parent plants.

Cross-pollination: Fertilization by the union of two gametes from different plants.

Embryo: The small plant contained within a mature seed.

F₁ hybrid: The first generation progeny of the cross of two nonisogenic plants.

Gene editing: A type of gene modification in which a nucleic acid molecule, such as DNA, is inserted, deleted or replaced in a native genome of a cell (such as a plant cell), for example using engineered nucleases which create site-specific double-strand breaks (DSBs) at desired locations in the genome, and whose improper repair by endogenous natural mechanisms results in an altered/non-native genomic sequence. Thus, the resulting genome is one that does not occur in nature. In some examples, gene editing results in the introduction of an exogenous transgene (e.g., one that does not occur naturally in the cell into which it is introduced) into the genome. In other examples, a gene-edited plant/plant part/cell provided herein is edited using an exogenous nucleic acid (e.g., gRNA) specific for StMYB93, thereby altering the endogenous sequence of StMYB93, but the exogenous nucleic acid molecule is not inserted into the genetic material (e.g., it disrupts the DNA target). In either case, such edited plants, plant parts, and cells are referred to as gene-edited plants, gene-edited plant parts, and gene-edited plant cells, respectively. The induced double-strand breaks are repaired through nonhomologous end-joining (NHEJ) or homologous recombination (HR), resulting in targeted mutations or repairs. CRISPR/Cas methods can be used to edit the sequence of one or more target genes, such as StMYB93. For example, gene editing in a plant can be used to confer a desirable trait to the plant, such as reduced sprouting.

Genetic inactivation/gene silencing/down-regulation: A general term describing epigenetic processes of gene regulation, including any technique or mechanism in which the expression of a gene is reduced or prevented. When used in reference to the expression of a nucleic acid molecule, such as a gene (e.g., StMYB93), refers to any process which results in a decrease in production of a gene product, such as a decrease of at least 20%, at least 50% or at least 75%. Gene silencing occurs when gene expression is significantly reduced (e.g., a reduction of at least 90%, at least 95%, or at least 99%) or even prevented. It can also be referred to as knocking out gene expression, when the gene is completely silenced. A gene product can be RNA or protein. Gene down-regulation or deactivation includes processes that decrease transcription of a gene or stability or translation of mRNA.

For example, a mutation, such as a substitution, partial or complete deletion, insertion, or other variation, can be made to a gene sequence that significantly reduces (and in some cases eliminates) production of the gene product or renders the gene product substantially or completely non-functional. For example, a genetic inactivation of the StMYB93 gene can produce a potato plant having a sprout suppressed phenotype. Genetic inactivation is also referred to herein as “functional deletion”.

Genotype. The genetic constitution of a cell, an organism, or an individual (i.e., the specific allele makeup of the individual) usually with reference to a specific character under consideration.

Isolated: An “isolated” biological component, such as a nucleic acid, protein or organelle, has been substantially separated or purified away from other biological components in the environment (such as a cell) in which the component occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

Light Sprout or Sprout. The “eyes” or sprouts that grow from the buds on the surface of the potato skin.

Locus/loci (plural): A position in the genome for a gene, SNP, mutation, etc., such as in a potato genome.

Plant: Includes reference to an immature or mature whole plant, including a plant from which seed, roots or leaves have been removed. Seed or embryo that will produce the plant are also considered to be the plant.

Plant Parts: Includes protoplasts, flowers, leaves, stems, roots, root tips, anthers, pistils, seeds, embryos, pollen, ovules, cotyledons, hypocotyl, flower, shoot, tissue, petiole, cells, calli, pods, meristematic cells, tuber, eye, light sprout, and the like. Includes plant cells of a tissue culture from which potato plants can be regenerated.

Progeny. Offspring; descendants. includes an F₁ potato plant produced from the cross of two potato plants where at least one plant includes potato cultivar ‘UNR-01’ and progeny further includes, but is not limited to, subsequent F₂, F₃, F₄, F₅, F₆, F₇, F₈, F₉, and F₁₀ generational crosses with the recurrent parental line.

Quantitative Trait Loci (QTL): Genetic loci that control to some degree numerically representable traits that are usually continuously distributed.

Regeneration. The development of a plant from tissue culture. The cells may, or may, not have been genetically modified. Plant tissue culture relies on the fact that all plant cells have the ability to generate a whole plant (totipotency). Single cells (protoplasts), pieces of leaves, or roots can be used to generate a new plant on culture media given the required nutrients and plant hormones.

Seed. The part of a flowering plant that typically contains the embryo with its protective coat and stored food and that can develop into a new plant under the proper conditions; fertilized and mature ovule.

Self-pollination: The transfer of pollen from the anther to the stigma of the same plant.

Single locus converted (conversion) plant: Plants developed by backcrossing and/or by genetic transformation, wherein essentially all (or all) of the desired morphological and physiological characteristics of a potato variety are recovered in addition to the characteristics of the single locus transferred into the variety via the backcrossing technique. In particular embodiments, a single locus conversion is generated by genome editing such as through use of engineered nucleases. Examples of engineered nucleases include, but are not limited to, Cas endonucleases (such as Cas9 or Cas13d), zinc finger nucleases (ZFNs), transcription activator-like effector nucleases (TALENs), and engineered meganucleases, also known as homing endonucleases. Naturally occurring nucleases can also find use for genome editing. In specific embodiments, endonucleases, both naturally occurring and engineered, may utilize any polypeptide-, DNA-, or RNA-guided genome editing systems.

Tissue culture: A composition that includes isolated cells of the same or a different type or a collection of such cells organized into parts of a plant.

Transformation: The introduction of new genetic material (e.g., exogenous transgenes) into plant cells. Exemplary mechanisms that are to transfer DNA into plant cells include (but not limited to) electroporation, microprojectile bombardment, Agrobacterium-mediated transformation and direct DNA uptake by protoplasts.

Transgene: A gene or genetic material that has been transferred into the genome of a plant, for example by genetic engineering methods. Exemplary transgenes include cDNA (complementary DNA) segment, which is a copy of mRNA (messenger RNA), and the gene itself residing in its original region of genomic DNA. In one example, describes a segment of DNA containing a gene sequence that is introduced into the genome of a potato plant or plant cell. This non-native segment of DNA may retain the ability to produce RNA or protein in the transgenic plant, or it may alter the normal function of the transgenic plant's genetic code. The transferred nucleic acid can be incorporated into the plant's germ line. Transgene can also describe any DNA sequence, regardless of whether it contains a gene coding sequence or it has been artificially constructed, which has been introduced into a plant or vector construct in which it was previously not found.

### New Potato Having Sprout Suppression

The present disclosure relates to a new potato variety ‘UNR-01’. Potato is one of the top four staple crops in the world and the number one vegetable crop in the US. However, up to 35% of the annual yield is lost each year during post-harvest storage. Premature sprouting and rots are the major culprits behind these losses. Through the unexpected phenotypes observed in ‘UNR-01’, including sprout suppression and unchanged wound healing capacities, these potatoes solve important postharvest storage issues. Moreover, ‘UNR-01’ potatoes do not require treatment with any chemical sprout inhibitors, that adds an advantage due to the recent ban on CIPC [isopropyl N-(3-chlorophenyl) carbamate], a major chemical sprout inhibitor widely used in the potato industry. Improving tuber storability can reduce post-harvest storage tuber losses, thereby improving food security for a major global staple crop.

The ‘UNR-01’ variety has the following phenotypes:
1. Sprout suppression for more than 14 months-Good for long term storability and food security
2. Less desiccation and shrinkage of tubers—Very good for any type of potato usage (frozen/fresh/chips) and maintaining yields by weight
3. Wound healing characteristics similar to wild type—Unchanged Curing Process
4. Reduced-sugar levels equal to wildtype tubers—Unchanged frying characteristics
5. A larger number of smaller tubers—Can be used as seed tubers or as fresh market “baby” potatoes.

‘UNR-01’ also has a genetic inactivation of StMYB93, by a deletion of about 1.2 kb of a StMYB93 gene.

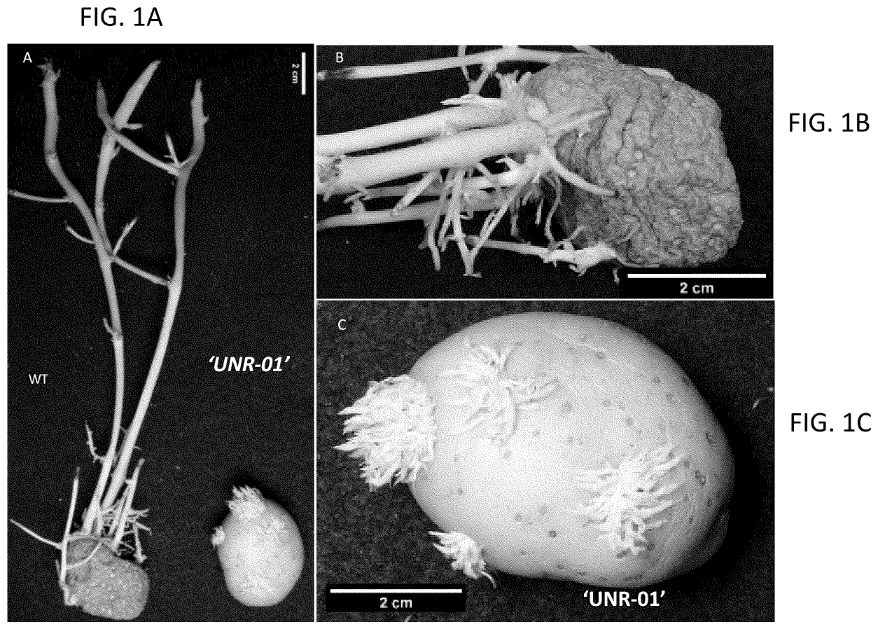

Thus, provided herein are plants and parts of potato variety ‘UNR-01’, wherein representative tuber, plantlet, or tissue culture of the variety will be deposited under the terms of the Budapest treaty. Also provided are bulk seed potatoes (e.g., a mixture of seed potatoes) containing ‘UNR-01’ seed potatoes. Also provided are compositions that include ‘UNR-01’ tubers, seed potatoes, or plants, and growth media, such as soil or a synthetic cultivation medium. The disclosure provides potato plants having or consisting of all of the morphological and physiological characteristics of ‘UNR-01’. The disclosure also provides potato plants having one or more of (such as at least two, at least three, at least four, at least five, or at least six) the morphological and physiological characteristics of ‘UNR-01’ (such as those shown in FIGS. 1-6 and 8). Also provided are seed potatoes and tubers of such plants, progeny of such plants (such as hybrids), parts of such plants (such as pollen, ovules, and cells). In one example, the disclosure provides potato plants having the genotype of ‘UNR-01’. For example, the disclosure provides plants produced by growing seed potatoes of the new potato variety ‘UNR-01’.

The disclosed ‘UNR-01’ plants and parts thereof can be used to produce other potato plants and seed potatoes, for example as part of a breeding program. Choice of breeding or selection methods using to generate new potato plants and seed potatoes can depend on the mode of plant reproduction, the heritability of the trait(s) being improved, and the type of variety used commercially (e.g., F₁ hybrid variety, inbred variety, etc.). For highly heritable traits, a choice of superior individual plants evaluated at a single location can be effective, whereas for traits with low heritability, selection can be based on mean values obtained from replicated evaluations of families of related plants. Exemplary selection methods commonly include pedigree selection, modified pedigree selection, mass selection, recurrent selection, and backcrossing. In some examples, ‘UNR-01’-derived plants generated, for example as part of a breeding program, have all of the morphological and physiological characteristics of ‘UNR-01’, for example in addition to one or more traits introduced into ‘UNR-01’. In some examples, ‘UNR-01’-derived plants include a single transgene and have all of the morphological and physiological characteristics of ‘UNR-01’, for example in addition to one or more traits introduced into ‘UNR-01’ by the transgene. In some examples, ‘UNR-01’-derived plants include a single locus conversion.

The complexity of inheritance can influence the choice of the breeding method. Backcross breeding can be used to transfer one or a few favorable genes for a highly heritable trait into a desirable variety. This approach has been used extensively for breeding disease-resistant varieties (e.g., see Bowers et al., 1992. Crop Sci. 32 (1): 67-72; Nickell and Bernard, 1992. Crop Sci. 32 (3): 835). Various recurrent selection techniques can be used to improve quantitatively inherited traits controlled by numerous genes.

Promising advanced breeding lines can be thoroughly tested and compared to appropriate standards in environments representative of the commercial target area(s), such as for three or more years. The best or most preferred lines are candidates for new commercial varieties. Those still deficient in certain traits may be used as parents to produce new populations for further selection.

One method of identifying a genetically superior plant is to observe its performance relative to other experimental plants and to one or more widely grown standard (such as commercially grown) varieties. Single observations can be generally inconclusive, while replicated observations provide a better estimate of genetic worth. Plant breeding can result in new, unique and superior potato varieties and hybrids from ‘UNR-01’. Two or more parental lines can be selected (wherein one is ‘UNR-01’), followed by repeated selfing and selection, producing many new genetic combinations. Each year, the germplasm to advance to the next generation is selected. This germplasm can be grown under unique and different geographical, climatic, and soil conditions, and further selections are then made, during and at the end of the growing season.

The development of new potato varieties from ‘UNR-01’ involves the development and selection of potato varieties, the crossing of these varieties, and selection of progeny from the superior hybrid crosses. A hybrid seed is produced by manual crosses between selected male-fertile parents or by using male sterility systems. Hybrids can be identified using certain single locus traits that indicate that the seed is truly a hybrid. A genetic locus conferring the traits may or may not be transgenic. Examples of such traits include, but are not limited to, male sterility, altered starch, thermotolerant amylase, herbicide resistance, resistance to bacterial, fungal, or viral disease, insect resistance, male fertility, restoration of male fertility, and enhanced nutritional quality. These genes are generally inherited through the nucleus but may be inherited through the cytoplasm. Some known exceptions to this are genes for male sterility, some of which are inherited cytoplasmically, but still act as a single locus trait. Additional data on parental lines as well as the phenotype of the hybrid can influence a decision whether to continue with the specific hybrid cross.

### Breeding of ‘UNR-01’

Breeding methods can be used with potato variety ‘UNR-01’ in the development of further potato plants. One such method includes using a ‘UNR-01’ potato plant, or a part thereof, utilizing said plant or plant part as a source of breeding material, and selecting a potato progeny plant with molecular markers in common with potato variety ‘UNR-01’ and/or with morphological and/or physiological characteristics selected from the characteristics listed in FIGS. 1-6 and 8. Breeding steps that may be used in the potato plant breeding program include pedigree breeding, backcrossing, mutation breeding, and recurrent selection. In conjunction with these steps, techniques such as RFLP-enhanced selection, genetic marker enhanced selection (for example, SSR markers), and the making of double haploids may be utilized.

Another method includes producing a population of potato variety ‘UNR-01’ progeny potato plants, by crossing potato variety ‘UNR-01’ with another potato plant, thereby producing a population of potato plants which derive 50% of their alleles from potato variety ‘UNR-01’. A plant of this population may be selected and repeatedly selfed or sibbed with a potato cultivar resulting from these successive filial generations. One example is the potato cultivar produced by this method and that has obtained at least 50% of its alleles from ‘UNR-01’. See, Milbourne, D., et al. “Comparison of PCR-based marker systems for the analysis of genetic relationships in cultivated potato” in Molecular Breeding. 3 (2): 127-136 (April 1997); Jacobs, J. M. E, et al., “genetic map of potato (Solanum tuberosum) integrating molecular markers, including transposons, and classical markers” Theoretical and Applied Genetics. 91 (2): 289-300 (July 1995).

One of ordinary skill in the art of plant breeding understands how to evaluate the traits of two plant varieties to determine if there is no significant difference between the two traits expressed by those varieties. For example, see, Fehr and Walt, Principles of Cultivar Development, pp. 261-286 (1987). Thus, provided are potato variety ‘UNR-01’ progeny potato plants that have a combination of at least two potato variety ‘UNR-01’ traits selected from the group consisting of those listed in FIGS. 1-6 and 8, so that said progeny potato plant is not significantly different for said traits than potato variety ‘UNR-01’ as determined at the 5% significance level when grown in the same environmental conditions. Using techniques described herein, molecular markers may be used to identify said progeny plant as a potato variety ‘UNR-01’ progeny plant. Mean trait values may be used to determine whether trait differences are significant, and the traits are measured on plants grown under the same environmental conditions. Once such a variety is developed, its value is substantial since it is important to advance the germplasm base as a whole in order to maintain or improve traits such as yield, disease resistance, pest resistance, and plant performance in extreme environmental conditions.

Progeny of potato variety ‘UNR-01’ can be characterized through their filial relationship with potato variety ‘UNR-01’, as for example, being within a certain number of breeding crosses of potato variety ‘UNR-01’. A breeding cross is a cross made to introduce new genetics into the progeny, and is distinguished from a self or a sib cross, which is made to select among existing genetic alleles. The lower the number of breeding crosses in the pedigree, the closer the relationship between potato variety ‘UNR-01’ and its progeny. For example, progeny produced by the methods described herein may be within 1, 2, 3, 4, or 5 breeding crosses of potato variety ‘UNR-01’.

Pedigree breeding and recurrent selection breeding methods can be used to develop varieties from breeding populations. Breeding programs combine desirable traits from two or more varieties or various broad-based sources into breeding pools from which varieties are developed by selfing and selection of desired phenotypes. Pedigree breeding starts with the crossing of two genotypes, such as ‘UNR-01’ and another potato variety having one or more desirable characteristics that is lacking or which complements ‘UNR-01’. If the two original parents do not provide all the desired characteristics, other sources can be included in the breeding population. In the pedigree method, superior plants are selfed and selected in successive filial generations.

In the succeeding filial generations, the heterozygous condition gives way to homogeneous varieties as a result of self-pollination and selection. Typically, in the pedigree method of breeding, five or more successive filial generations of selling and selection is practiced: F₁ to F₂: F₂ to F₃: F₃ to F₄; F₄ to F₅; etc. After a sufficient amount of inbreeding, successive filial generations will serve to increase seed of the developed variety.

Two parents (e.g., wherein one of the parents is ‘UNR-01’) which possess favorable, complementary traits are crossed to produce an F₁. An F₂ population is produced by selfing one or several F₁s, such as an F₁ having a low harvest moisture level. Selection of the best or most preferred individuals can begin in the F₂ population (or later depending upon the breeding objectives); then, beginning in the F₃, the best or most preferred individuals in the best families can be selected. Replicated testing of families can begin in the F₃ or F₄ generation to improve the effectiveness of selection for traits with low heritability. At an advanced stage of inbreeding (e.g., F₆ and F₇), the best lines or mixtures of phenotypically similar lines can be tested for potential commercial release as new varieties.

Mass and recurrent selections can be used to improve populations of either self- or cross-pollinating crops. A genetically variable population of heterozygous individuals is either identified or created by intercrossing several different parents. The best or most preferred plants are selected based on individual superiority, outstanding progeny, or excellent combining ability. The selected plants are intercrossed to produce a new population in which further cycles of selection are continued.

Backcross breeding has been used to transfer genetic loci for simply inherited, highly heritable traits into a desirable homozygous variety which is the recurrent parent (e.g., ‘UNR-01’). The goal of a backcross protocol is to alter or substitute a single trait or characteristic in the original variety. The source of the trait to be transferred is called the donor or nonrecurrent parent. The resulting plant is typically expected to have the attributes of the recurrent parent (e.g., variety) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., ‘UNR-01’) and the desirable trait transferred from the donor parent. This is also known as single gene conversion.

In addition to being used to create a backcross conversion, backcrossing can also be used in combination with pedigree breeding. Backcrossing can be used to transfer one or more specifically desirable traits from one variety, the donor parent, to a developed variety called the recurrent parent, which has overall good agronomic characteristics yet lacks that desirable trait or traits. However, the same procedure can be used to move the progeny toward the genotype of the recurrent parent, but at the same time retain many components of the nonrecurrent parent by stopping the backcrossing at an early stage and proceeding with selfing and selection. For example, a potato variety may be crossed with another variety to produce a first-generation progeny plant. The first-generation progeny plant may then be backcrossed to one of its parent varieties to create a BC1 or BC2. Progeny are selfed and selected so that the newly developed variety has many of the attributes of the recurrent parent and yet several of the desired attributes of the nonrecurrent parent. This approach leverages the value and strengths of the recurrent parent for use in new potato varieties.

Therefore, provided is a method of making a backcross conversion of ‘UNR-01’, by crossing a plant of ‘UNR-01’ with a donor plant having a desired trait, selecting an F₁ progeny plant comprising the desired trait, and backcrossing the selected F₁ progeny plant to a plant of ‘UNR-01’ to produce BC1, BC2, BC3, etc. This method may further include obtaining a molecular marker profile of ‘UNR-01’ and using the molecular marker profile to select for a progeny plant with the desired trait and the molecular marker profile of ‘UNR-01’. In one example, the desired trait is a mutant gene, gene, or transgene present in the donor parent.

Recurrent selection is a method used to improve a population of plants. Potato variety ‘UNR-01’ is suitable for use in a recurrent selection program. The method can include individual plants cross pollinating with each other to form progeny. The progeny are grown and the superior progeny selected by any number of selection methods, which include individual plant, half-sib progeny, full-sib progeny, and selfed progeny. The selected progeny are cross pollinated with each other to form progeny for another population. This population is planted and again superior plants are selected to cross pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use, including the production of a synthetic cultivar. A synthetic cultivar is the resultant progeny formed by the intercrossing of several selected varieties.

Mass selection is a useful technique when used in conjunction with molecular marker enhanced selection. In mass selection, seeds from individuals are selected based on phenotype or genotype. These selected seeds are then bulked and used to grow the next generation. Bulk selection requires growing a population of plants in a bulk plot, allowing the plants to self-pollinate, harvesting the seed in bulk, and then using a sample of the seed harvested in bulk to plant the next generation. Also, instead of self-pollination, directed pollination could be used as part of the breeding program.

Mass and recurrent selections can be used to improve populations of either self- or cross-pollinating crops. A genetically variable population of heterozygous individuals is either identified, or created, by intercrossing several different parents. The plants are selected based on individual superiority, outstanding progeny, or excellent combining ability. The selected plants are intercrossed to produce a new population in which further cycles of selection are continued.

Methods for crossing the new potato variety ‘UNR-01’ with itself or a second plant are provided, as are the seeds and plants produced by such methods, including F₁ and F₂ plants and seeds. Such methods can be used for propagation of the new potato variety ‘UNR-01’, or can be used to produce hybrid potato seeds and the plants grown therefrom. Hybrid potato plants can be used, for example, in the commercial production of potato products or in breeding programs for producing novel potato varieties. A hybrid plant can also be used as a recurrent parent at any given stage in a backcrossing protocol during the production of a single locus conversion (for example introduction of one or more desirable traits) of the new potato variety ‘UNR-01’. Exemplary breeding methods that can be used are found in e.g., Allard, Principles of Plant Breeding (1960); Simmonds, Principles of Crop Improvement (1979); Sneep, et al. (1979); Cooper, S. G., D. S. Douches and E. J. Grafius. 2004. Genetic engineering and traditional breeding can be used or combined.

Methods of producing potato plants and/or seed are provided. Such a method can include crossing the new potato variety ‘UNR-01’ with itself or a second potato plant and harvesting resulting potatoes, such as an F₁ hybrid potatoes. The resulting plant can be grown, resulting in an F₁ potato plant or part thereof.

In one example methods of producing an inbred potato plant derived from potato variety ‘UNR-01’ are provided. In one example such methods include (a) preparing a progeny plant derived from potato variety ‘UNR-01’ by crossing a plant of potato variety ‘UNR-01’ with a potato plant of a second variety; (b) crossing the progeny plant with itself or a second plant to produce a seed of a progeny plant of a subsequent generation; (c) growing a progeny plant of a subsequent generation (for example from seed potatoes) and crossing the progeny plant of a subsequent generation with itself or a second plant; and (d) repeating steps (b) and (c) for an additional generation (such as at least 2, at least 3, at least 4, at least 5, at least 6, at least 7, at least 8 at least 9, at least 10, at least 15 or at least 20, such as 2 to 10, 3 to 10, or 3 to 15 generations, such as 2, 3, 4, 5, 6, 7, 8, 9, 10, 15, 20, or 30 generations) with sufficient inbreeding to produce an inbred potato plant derived from the potato variety ‘UNR-01’.

The second plant crossed with the new potato variety ‘UNR-01’ for the purpose of developing novel potato varieties is typically a plant which either itself exhibits one or more additional characteristics or which exhibits one or more additional characteristic(s) when in hybrid combination. In one example, the second potato plant is transgenic. Exemplary additional characteristics include, but are not limited to: herbicide tolerance (or resistance), resistance to a bacterial disease, resistance to a viral disease, resistance to an insect, resistance to a fungal disease, resistance to a nematode, resistance to a pest, male sterility, site-specific recombination, abiotic stress tolerance, modified linolenic or linoleic acid content, modified antioxidant characteristics, modified essential seed amino acid characteristics, modified fatty acid metabolism, modified carbohydrate metabolism, modified phytic acid metabolism, modified protein metabolism, water stress resistance, restoration of male fertility, altered starch, thermotolerant amylase or other improved nutritional qualities.

When the new potato variety ‘UNR-01’ is crossed with another different variety, first generation (F₁) potato progeny are produced. The hybrid progeny are produced regardless of characteristics of the two varieties crossed. As such, an F₁ hybrid potato plant can be produced by crossing ‘UNR-01’ with any second potato plant. The second potato plant can be genetically homogeneous (e.g., inbred) or can itself be a hybrid. Therefore, the disclosure provides any F₁ hybrid potato plant produced by crossing the new potato variety ‘UNR-01’ with a second potato plant (such as a transgenic plant having one or more genes that confer to the plant one or more additional characteristics).

Potato plants can be crossed by either natural or mechanical techniques. Natural pollination can occur by natural cross pollination, which typically is aided by wind. Mechanical pollination can be accomplished either by controlling the types of pollen that can blow or by pollinating by hand.

One use of the instant potato variety is in the production of hybrid potatoes. Any time the potato plant ‘UNR-01’ is crossed with a different potato plant, a potato hybrid plant is produced. As such, a hybrid potato plant can be produced by crossing ‘UNR-01’ with any second potato plant. Essentially any other potato plant can be used to produce a potato plant having potato plant ‘UNR-01’ as one parent. All that is required is that the second plant be fertile, and not be ‘UNR-01’. Thus, any potato plant produced using potato plant ‘UNR-01’ is within the scope of this disclosure. As used herein, crossing can mean selfing, backcrossing, crossing to another or the same variety, crossing to populations, and the like.

The goal of producing an F₁ hybrid is to manipulate the genetic complement of potato to generate new combinations of genes which interact to yield new or improved traits (phenotypic characteristics). A process of producing an F₁ hybrid typically begins with the production of one or more inbred plants. Those plants are produced by repeated crossing of ancestrally related potato plants to try to combine certain genes within the inbred plants.

New inbred varieties can be generated from ‘UNR-01’. Novel varieties may be created by crossing ‘UNR-01’, followed by multiple generations of breeding. New varieties may be created by crossing ‘UNR-01’ with any second potato plant. In selecting such a second plant to cross for the purpose of developing novel inbred lines, it may be desired to choose those plants which either themselves exhibit one or more desirable characteristics or which exhibit the desirable characteristic(s) when in hybrid combination. Exemplary desirable characteristics include greater yield, reduced harvest moisture content, resistance to insecticides, herbicides, pests, and disease, tolerance to heat and drought, reduced time to crop maturity, better agronomic quality, higher nutritional value, improved wound healing characteristics, reduced desiccation and shrinkage of tubers, and uniformity in germination times.

The pedigree breeding method involves crossing two genotypes. Each genotype can have one or more desirable characteristics lacking in the other; or, each genotype can complement the other. If the two original parental genotypes do not provide all of the desirable characteristics, other genotypes can be included in the breeding population. Superior plants that are the products of these crosses are selfed and selected in successive generations. Each succeeding generation becomes more homogeneous as a result of self-pollination and selection. Typically, this method of breeding involves five or more generations of selfing and selection. After at least five generations, the inbred plant is considered genetically pure.

The development of inbred plants generally requires at least about five to seven generations of selfing. Inbred plants are then crossbred to develop improved F₁ hybrids. Hybrids are then screened and evaluated in small scale field trials. Typically, about 10 to 15 phenotypic traits, selected for their potential commercial value, are measured. A selection index of the most commercially important traits is used to help evaluate hybrids.

During the next several years, a progressive elimination of hybrids occurs based on more detailed evaluation of their phenotype. Eventually, trials are conducted to formally compare the experimental hybrids being developed with other hybrids, some of which were previously developed and generally are commercially successful. That is, comparisons of experimental hybrids are made to competitive hybrids to determine if there was any advantage to further development of the experimental hybrids. After comparison testing is complete, determinations may be made whether commercial development should proceed for a given hybrid.

The present disclosure provides a genetic complement of the potato variety ‘UNR-01’. As used herein, the phrase “genetic complement” means an aggregate of nucleotide sequences, the expression of which defines the phenotype of a potato plant or a cell or tissue of that plant. By way of example, a potato plant is genotyped to determine a representative sample of the inherited markers it possesses. Markers are alleles at a single locus. They are preferably inherited in codominant fashion so that the presence of both alleles at a diploid locus is readily detectable, and they are free of environmental variation, i.e., their heritability is 1. This genotyping can be performed on at least one generation of the descendant plant for which the numerical value of the quantitative trait or traits of interest are also determined. The array of single locus genotypes is expressed as a profile of marker alleles, two at each locus. The marker allelic composition of each locus can be either homozygous or heterozygous. Homozygosity is a condition in which both alleles at a locus are characterized by the same nucleotide sequence or size of a repeated sequence. Heterozygosity refers to different conditions of the gene at a locus. An exemplary type of genetic marker for use with the invention is simple sequence repeats (SSRs), although potentially any other type of genetic marker could be used, for example, restriction fragment length polymorphisms (RFLPs), amplified fragment length polymorphisms (AFLPs), single nucleotide polymorphisms (SNPs), and isozymes.

Single seed descent of ‘UNR-01’ can also be used, which refers to planting a segregating population, harvesting a sample of one seed per plant, and using the one-seed sample to plant the next generation. When the population has been advanced from the F₂ to the desired level of inbreeding, the plants from which lines are derived will each trace to different Fa individuals. The number of plants in a population declines each generation due to failure of some seeds to germinate or some plants to produce at least one seed. As a result, not all F₂ plants originally sampled in the population will be represented by a progeny when generation advance is completed.

Mutation breeding is another method of introducing new traits into potato ‘UNR-01’. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation; such as X-rays. Gamma rays (e.g., cobalt 60 or cesium 137), neutrons. (product of nuclear fission by uranium 235 in an atomic reactor). Beta radiation (emitted from radioisotopes such as phosphorus 32 or carbon 14), or ultraviolet radiation (e.g., from 2500 to 2900 nm), or chemical mutagens (e.g., base analogues (5-bromo-uracil)), related compounds (8-ethoxy caffeine), antibiotics (streptonigrin), alkylating agents (sulfur mustards, nitrogen mustards, epoxides, ethylenamines, sulfates, sulfonates, sulfones, lactones), azide, hydroxylamine, nitrous acid, or acridines. Once a desired trait is observed through mutagenesis the trait may then be incorporated into existing germplasm by traditional breeding techniques. Details of mutation breeding can be found in Fehr, “Principles of Cultivar Development,” Macmillan Publishing Company (1993). In addition, mutations created in other potato plants may be used to produce a backcross conversion of potato ‘UNR-01’ that includes such mutation.

The production of double haploids can be used for the development of plants with a homozygous phenotype in the breeding program. For example, a potato plant for which potato ‘UNR-01’ is a parent can be used to produce double haploid plants. Double haploids are produced by the doubling of a set of chromosomes (IN) from a heterozygous plant to produce a completely homozygous individual. For example, see, Rokka, V. N. “Potato haploids in Breeding” in A. Touracv et al. (eds.) Advances in Haploid Production in Higher Plants, Spring Science+Business Media B.V. (2009), Chapter 17; and De Maine, M. J. “Potato Haploid Technologies” in M. Maluszynski et al. (eds), Doubled Haploid Production in Crop Plants, pp 241-247 (2003). Thus, provided is a process for making a substantially homozygous potato ‘UNR-01’ progeny plant by producing or obtaining a seed from the cross of potato ‘UNR-01’ and another potato plant and applying double haploid methods to the F₁ seed or F₁ plant or to any successive filial generation.

In particular, a process of making seed retaining the molecular marker profile of potato ‘UNR-01’ is provided, which can include obtaining or producing F₁ seed for which potato ‘UNR-01’ is a parent, inducing doubled haploids to create progeny without the occurrence of meiotic segregation, obtaining the molecular marker profile of potato ‘UNR-01’, and selecting progeny that retain the molecular marker profile of potato ‘UNR-01’.

### Potato Plants Having One or More Additional Heritable Traits

The disclosure provides plants of the new potato variety ‘UNR-01’ modified to include one or more additional heritable traits, such as 1, 2, 3 4 or 5 heritable traits. In some examples, such plants can be developed using backcrossing or genetic engineering (for example by introducing one or more transgenes into the ‘UNR-01’ variety, wherein the transgenes encode one or more additional traits), wherein in some examples essentially all or all of the morphological and physiological characteristics of the ‘UNR-01’ variety are recovered (including sprout suppression) in addition to a genetic locus transferred into the plant via backcrossing. In one example, a single genetic locus transferred into the plant via backcrossing or genetic engineering. Plants developed using such methods can be referred to as a single locus converted plant.

In one example, the method of introducing one or more additional traits into potato variety ‘UNR-01’ includes (a) crossing a plant of variety ‘UNR-01’ with a second plant having one or more additional traits to produce F₁ progeny plants; (b) selecting F₁ progeny plants that have the one or more additional traits to produce selected F₁ progeny plants; (c) crossing the selected progeny plants with at least a first plant of variety ‘UNR-01’ to produce backcross progeny plants; (d) selecting backcross progeny plants that have the one or more additional traits and in some examples also all of the physiological and morphological characteristics of potato variety ‘UNR-01’ to produce selected backcross progeny plants; and (e) repeating steps (c) and (d) one or more times in succession to produce selected second or higher backcross progeny plants that have the one or more additional traits and in some examples also all of the physiological and morphological characteristics of potato variety ‘UNR-01’ (including sprout suppression) when grown in the same environmental conditions.

Backcrossing methods can be used to improve or introduce a characteristic into the new potato variety ‘UNR-01’ (e.g., see The Potato Genome Sequencing Consortium, “Genome sequence and analysis of the tuber crop potato” Nature. 475:189-195, 2011; Hallauer, et al., “Corn Breeding,” Corn and Corn Improvements, 18:463-481, 1988). The parental plant which contributes the locus for the additional characteristic is termed the “nonrecurrent” or “donor” parent. This terminology refers to the fact that the nonrecurrent parent is used one time in the backcross protocol and therefore does not recur. The parental potato plant to which the locus or loci from the nonrecurrent parent are transferred the recurrent parent as it is used for several rounds in the backcrossing protocol. In a typical backcross protocol, the original variety of interest (recurrent parent, e.g., ‘UNR-01’) is crossed to a second variety (nonrecurrent parent) that carries the single locus of interest (such as a desirable trait) to be transferred. The resulting progeny from this cross are then crossed again to the recurrent parent and the process is repeated until a potato plant is obtained wherein essentially all or all of the morphological and physiological characteristics of the recurrent parent (e.g., ‘UNR-01’) are recovered (such as sprout suppression) in the converted plant, in addition to the single transferred locus from the nonrecurrent parent.

Both naturally occurring and transgenic DNA sequences may be introduced through backcrossing techniques. A backcross conversion may produce a plant with a trait or locus conversion in at least two or more backcrosses, including at least 2 crosses, at least 3 crosses, at least 4 crosses, at least 5 crosses, and the like. Molecular marker assisted breeding or selection may be utilized to reduce the number of backcrosses necessary to achieve the backcross conversion. For example, see, Barone, Amalia, “Molecular marker-assisted selection for potato breeding” American Journal of Potato Research. 81 (2): 111-117 (March 2004), and Openshaw, S. J., et al., Marker-assisted Selection in Backcross Breeding, Proceedings Symposium of the Analysis of Molecular Data, Crop Science Society of America, Corvallis, Oreg. (August 1994), where it is demonstrated that a backcross conversion can be made in as few as two backcrosses.

The complexity of the backcross conversion method depends on the type of trait being transferred (single genes or closely linked genes as compared to unlinked genes), the level of expression of the trait, the type of inheritance (cytoplasmic or nuclear), and the types of parents included in the cross. For single gene traits that are relatively easy to classify, the backcross method is effective and relatively easy to manage. Desired traits that may be transferred through backcross conversion include, but are not limited to, sterility (nuclear and cytoplasmic), fertility restoration, nutritional enhancements, drought tolerance, nitrogen utilization, altered fatty acid profile, low phytate, industrial enhancements, disease resistance (bacterial, fungal, or viral), insect resistance, and herbicide resistance. In addition, an introgression site itself, such as an FRT site, Lox site, or other site-specific integration site, may be inserted by backcrossing and utilized for direct insertion of one or more genes of interest into a specific plant variety. In some embodiments, the number of loci that may be backcrossed into potato cultivar ‘UNR-01’ is at least 1, 2, 3, 4, or 5, and/or no more than 6, 5, 4, 3, or 2, such as 1, 2, 3, 4, 5, or 6 loci. A single locus may contain several transgenes, such as a transgene for disease resistance that, in the same expression vector, also contains a transgene for herbicide resistance. The gene for herbicide resistance may be used as a selectable marker and/or as a phenotypic trait. A single locus conversion of site-specific integration system allows for the integration of multiple genes at the converted loci.

The backcross conversion may result from either the transfer of a dominant allele or a recessive allele. Selection of progeny containing the trait of interest is accomplished by direct selection for a trait associated with a dominant allele. Transgenes transferred via backcrossing typically function as a dominant single gene trait and are relatively easy to classify. Selection of progeny for a trait that is transferred via a recessive allele requires growing and selfing the first backcross generation to determine which plants carry the recessive alleles. Recessive traits may require additional progeny testing in successive backcross generations to determine the presence of the locus of interest. The last backcross generation is usually selfed to give pure breeding progeny for the gene(s) being transferred, although a backcross conversion with a stably introgressed trait may also be maintained by further backcrossing to the recurrent parent with selection for the converted trait.

Along with selection for the trait of interest, progeny are selected for the phenotype of the recurrent parent. The backcross is a form of inbreeding, and the features of the recurrent parent are automatically recovered after successive backcrosses. Poehlman, Breeding Field Crops, p. 204 (1987). Poehlman suggests from one to four or more backcrosses, but as noted above, the number of backcrosses necessary can be reduced with the use of molecular markers. Other factors, such as a genetically similar donor parent, may also reduce the number of backcrosses necessary. As noted by Poehlman, backcrossing is easiest for simply inherited, dominant, and easily recognized traits.

One process for adding or modifying a trait or locus in potato ‘UNR-01’ includes crossing potato ‘UNR-01’ plants grown from potato ‘UNR-01’ seed with plants of another potato variety that include the desired trait or locus, selecting F₁ progeny plants that comprise the desired trait or locus to produce selected F₁ progeny plants, crossing the selected progeny plants with the potato ‘UNR-01’ plants to produce backcross progeny plants, selecting for backcross progeny plants that have the desired trait or locus and in some examples also all of the morphological characteristics of potato ‘UNR-01’ to produce selected backcross progeny plants, and backcrossing to potato ‘UNR-01’ three or more times in succession to produce selected fourth or higher backcross progeny plants that comprise said trait or locus. The modified potato ‘UNR-01’ may be further characterized as having all of the physiological and morphological characteristics of potato ‘UNR-01’ listed herein as determined at the 5% significance level when grown in the same environmental conditions and/or may be characterized by percent similarity or identity to potato ‘UNR-01’ as determined by SSR markers. The above method may be utilized with fewer backcrosses in appropriate situations, such as when the donor parent is highly related or markers are used in the selection step. Desired traits that may be used include those nucleic acids known in the art, some of which are listed herein, that will affect traits through nucleic acid expression or inhibition. Desired loci include the introgression of FRT, Lox, and other sites for site specific integration, which may also affect a desired trait if a functional nucleic acid is inserted at the integration site.

In addition, the above process and other similar processes described herein may be used to produce first generation progeny potato seed by adding a step at the end of the process that includes crossing potato ‘UNR-01’ with the introgressed trait or locus with a different potato plant and harvesting the resultant first-generation progeny potato seed.

Direct selection can be applied where the single locus acts as a dominant trait. An example of a dominant trait is the herbicide resistance trait (such as glyphosate tolerance). For the selection process, the progeny of the initial cross are sprayed with an herbicide (such as glyphosate) prior to the backcrossing. The spraying eliminates any plants which do not have the herbicide tolerance characteristic; only those plants which have the herbicide resistance gene are used in the subsequent backcross. This process is then repeated for all additional backcross generations.

Selection of plants for breeding may not be dependent on the phenotype of a plant and instead can be based on genetic investigations. For example, a suitable genetic marker can be used which is closely genetically linked to a trait. One of these markers can therefore be used to identify the presence or absence of a trait in the offspring of a particular cross, and hence can be used in selection of progeny for continued breeding. This technique is referred to as marker assisted selection. Any other type of genetic marker or other assay which identifies the relative presence or absence of a trait of interest in a plant can also be useful for breeding. Procedures for marker assisted selection applicable to the breeding of potatoes are known. Such methods can be useful in the case of recessive traits and variable phenotypes, or where conventional assays are more expensive, time consuming or otherwise disadvantageous. Types of genetic markers, which includes markers identified through the use of techniques such as Isozyme Electrophoresis, Restriction Fragment Length Polymorphisms (RFLPs), Simple Sequence Length Polymorphisms (SSLPs), Simple Sequence Repeats (SSRs), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs) (EP 534 858), Quantitative Trait Loci (QTL) mapping, and Single Nucleotide Polymorphisms (SNPs), can be used in plant breeding methods utilizing ‘UNR-01’.

Qualitative characters can be useful as phenotype-based genetic markers in potatoes; however, some or many may not differ among varieties commonly used as parents.

Useful or desirable traits can be introduced by backcrossing, as well as directly into a plant by genetic transformation methods. Genetic transformation can therefore be used to insert a selected transgene into the ‘UNR-01’ variety or can, alternatively, be used for the preparation of transgenes which can be introduced by backcrossing. Thus, the disclosure provides methods of producing a plant of potato variety ‘UNR-01’ that includes one or more added traits, for example that include introducing a transgene(s) conferring the one or more additional traits into a plant of potato variety ‘UNR-01’ (for example by transformation with a transgene that confers upon the potato plant the additional trait), thereby producing a plant of potato variety ‘UNR-01’ that includes the one or more added traits. In some embodiments, a transgenic variant of potato variety ‘UNR-01’ contains at least one transgene (such as a single transgene) and has all of the physiological and morphological characteristics of ‘UNR-01’ in addition to the trait conferred by the transgene.

In one example, the transgene encodes one or genes that confer herbicide resistance, insect resistance, disease resistance, decreased phytate, or modified fatty acid or carbohydrate metabolism, such as a polynucleotide conferring resistance to imidazolinone, dicamba, sulfonylurea, glyphosate, glufosinate, triazine, PPO-inhibitor herbicides, benzonitrile, cyclohexanedione, phenoxy proprionic acid, and L-phosphinothricin; a polynucleotide encoding a Bacillus thuringiensis polypeptide; a polynucleotide encoding phytase, FAD-2, FAD-3, galactinol synthase, or a raffinose synthetic enzyme; or a polynucleotide conferring resistance to Phytophthora late blight, Alternaria early blight, Erwinia soft rot, Streptomyces common scab, Spongospora powdery scab, Fusarium dry rot, Potato Leaf Roll Virus (PLRV), Globodera rostochiensis, or Globodera pallida.

Methods for the transformation of plants, including potatoes, are known. See, for example, Miki et al., “Procedures for Introducing Foreign DNA into Plants,” in Methods in Plant Molecular Biology and Biotechnology, Glick and Thompson Eds., CRC Press, Inc., Boca Raton, pp. 67-88 (1993), and Armstrong, “The First Decade of Maize Transformation: A Review and Future Perspective,” Maydica, 44:101-109 (1999) and Chakaravarty. B., et al., American Journal of Potato Research 84 (4): 301-311. In addition, expression vectors and in vitro culture methods for plant cell or tissue transformation and regeneration of plants are available. See, for example, Gruber, et al., “Vectors for Plant Transformation,” in Methods in Plant Molecular Biology and Biotechnology, Glick and Thompson Eds., CRC Press, Inc., Boca Raton, pp. 89-119 (1993).

Methods for introducing a nucleic acid molecule (e.g., transgene), such as DNA, RNA, or inhibitory RNAs, or vectors that include such, are known, and the disclosure is not limited to particular methods. Exemplary techniques which can be employed for the genetic transformation of include, but are not limited to, electroporation (U.S. Pat. No. 5,384,253), electrotransformation (U.S. Pat. No. 5,371,003), microprojectile bombardment (U.S. Pat. Nos. 5,550,318, 5,736,369 and 5,538,880; and PCT Publication WO 95/06128), Agrobacterium-mediated transformation (U.S. Pat. No. 5,591,616 and European Patent Application Publication No. EP0672752), DNA injection, direct DNA uptake transformation of protoplasts and silicon carbide fiber-mediated transformation (U.S. Pat. Nos. 5,302,532 and 5,464,765).

To effect transformation by electroporation, friable tissues, such as a suspension culture of cells or embryogenic callus, can be used. Alternatively, immature embryos or other organized tissue can be transformed directly. Protoplasts can also be employed for electroporation transformation of plants (Bates. 1994. Mol. Biotechnol. 2 (2): 135-145; Lazzeri. 1995. Methods Mol. Biol. 49:95-106).

A generally applicable method of plant transformation is microprojectile-mediated transformation where DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. Sanford et al., Part. Sci. Technol., 5:27 (1987); Sanford, Trends Biotech., 6:299 (1988); Klein, et al., Bio/Tech., 6:559-563 (1988); Sanford, Physiol Plant, 7:206 (1990); Klein, et al., Biotechnology, 10:268 (1992). See also, U.S. Pat. Nos. 5,015,580 and 5,322,783.

Another method for physical delivery of DNA to plants is sonication of target cells. Zhang, et al., Bio/Technology, 9:996 (1991). Alternatively, liposome and spheroplast fusion have been used to introduce expression vectors into plants. Deshayes, et al., EMBO J., 4:2731 (1985); Christou, et al., Proc Natl. Acad. Sci. USA, 84:3962 (1987). Direct uptake of DNA into protoplasts using CaCl2) precipitation, polyvinyl alcohol or poly-L-ornithine have also been reported. Hain, et al., Mol. Gen. Genet., 199:161 (1985) and Draper, et al., Plant Cell Physiol., 23:451 (1982). Electroporation of protoplasts and whole cells and tissues has also been described (D'Halluin, et al., Plant Cell, 4:1495-1505 (1992); and Spencer, et al., Plant Mol. Biol., 24:51-61 (1994)).

Agrobacterium-mediated transfer can be used to introduce gene loci into plant cells (See, for example, Orozco-Cárdenas, M. L., et al., (2014). Potato (Solanum tuberosum L.) Methods in Molecular biology, Agrobacterium Protocols edited by Kan Wang. Third Edition Volume 2, Humana Press. Totowa, N.J., (2014)).

Following transformation of potato target tissues, expression of selectable marker genes allows for preferential selection of transformed cells, tissues, and/or plants, using regeneration and selection methods.

Transformation of plant protoplasts can also be achieved using methods based on calcium phosphate precipitation, polyethylene glycol treatment, electroporation, and combinations of these treatments (e.g., Potrykus et al. 1985. Mol. Gen. Genet. 199 (2): 169-177; Omirulleh et al. 1993. Plant Mol. Biol. 21 (3): 415-428; Fromm et al. 1986. Nature. 319 (6056): 791-739; Uchimiya et al. 1986. Mol. Gen. Genet. 204 (2): 207-207; Marcotte et al. 1988. Nature 335 (6189): 454-457).

Included among various plant transformation techniques are methods permitting the site-specific modification of a plant genome. These modifications can include, but are not limited to, site-specific mutations, deletions, insertions, and replacements of nucleotides. These modifications can be made anywhere within the genome of a plant, for example, in genomic elements, including, among others, coding sequences, regulatory elements, and non-coding DNA sequences. Any number of such modifications can be made and the modifications may be made in any order or combination, for example, simultaneously, all together, or one after another. Such methods may be used to modify a particular trait conferred by a locus. Techniques for making such modifications in ‘UNR-01’ by genome editing are well known in the art and include, for example, use of CRISPR-Cas systems (e.g., CRISPR/Cas9 or CRISPR/Cas13d), engineered homing endonucleases/meganucleases (EMNs), zinc-finger nucleases (ZFNs), and transcription activator-like effector nucleases (TALENs), among others. Gene editing can also be done using crRNA-guided surveillance systems for gene editing.

In some embodiments, a transgenic variant of potato ‘UNR-01’ includes at least one transgene, such as 1, 2, 3, 4 or 5 transgenes. In some examples, a transgene includes various genetic elements can be introduced into the plant genome using transformation. These elements include, but are not limited to, genes, coding sequences, inducible, constitutive and tissue specific promoters, enhancing sequences, and signal and targeting sequences. For example, see the traits, genes, and transformation methods listed in U.S. Pat. No. 6,118,055.

It is understood to those of skill in the art that a transgene need not be directly transformed into a plant, as techniques for producing stably transformed potato plants that pass single loci to progeny by Mendelian inheritance are known. Such loci may therefore be passed from parent plant to progeny plants by standard plant breeding techniques.

Transformation of ‘UNR-01’ can use an expression vector which will function in plant cells. Such a vector includes DNA encoding a gene under control of, or operatively linked to, a regulatory element (for example, a promoter). Expression vectors include at least one genetic marker operably linked to a regulatory element (for example, a promoter) that allows transformed cells containing the marker to be either recovered by negative selection, i.e., inhibiting growth of cells that do not contain the selectable marker gene, or by positive selection, i.e., screening for the product encoded by the genetic marker. Exemplary selectable marker genes for plant transformation include genes that code for enzymes that metabolically detoxify a selective chemical agent which may be an antibiotic or an herbicide, or genes that encode an altered target which is insensitive to the inhibitor. Positive selection methods can be used to identify transformed cells and plants. One exemplary selectable marker gene for plant transformation is neomycin phosphotransferase II (np (II) which, when under the control of plant regulatory signals, confers resistance to kanamycin. Another exemplary selectable marker gene is hygromycin phosphotransferase which confers resistance to the antibiotic hygromycin. Additional selectable marker genes include Pain 1-9a and Pain1-8c which both correspond to the group a alleles of the vacuolar acid invertase gene; PainIprom-d/e; Stp23-8b, StpL-3b, and StpL-3e which originate from two plastid starch phosphorylase genes; AGPsS-9a which is positively associated an increase in tuber starch content, starch yield and chip quality, and AGPsS-10a which is associated with a decrease in the average tuber starch content, starch yield and chip quality; GP171-a which corresponds to allele 1a of ribulose bisphosphate carboxylase activase; and Rca-1a. See Li, Li, et al, “Validation of candidate gene markers for marker-assisted selection of potato cultivars with improved tuber quality” Theor Appl Genet. 2013 April: 126 (4): 1039-1052. Selectable marker genes for plant transformation not of bacterial origin include, for example, mouse dihydrofolate reductase, plant 5-enolpyruvylshikimate-3-phosphate synthase, and plant acetolactate synthase (Eichholtz, et al., Somatic Cell Mol. Genet., 13:67 (1987): Shah, et al., Science, 233:478 (1986); Charest, et al., Plant Cell Rep., 8:643 (1990)). Another class of marker genes for plant transformation requires screening of presumptively transformed plant cells, rather than direct genetic selection of transformed cells, for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Exemplary marker genes for screening presumptively transformed cells include β-glucuronidase (GUS), β-galactosidase, luciferase, and chloramphenicol acetyltransferase.

Expression of genes in expression vectors is driven by a nucleotide sequence comprising a regulatory element (for example, a promoter). A promoter includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters that initiate transcription only in a certain tissue are referred to as “tissue-specific.” A “cell-type” specific promoter primarily drives expression in certain cell types in one or more organs, for example, vascular cells in roots or leaves. An “inducible” promoter is a promoter which is under environmental control. Examples of environmental conditions that may affect transcription by inducible promoters include anaerobic conditions or the presence of light. Tissue-specific, tissue-preferred, cell-type specific, and inducible promoters constitute the class of “non-constitutive” promoters. A “constitutive” promoter is a promoter that is active under most environmental conditions. An inducible promoter can be operably linked to a gene for expression in potatoes. Optionally, the inducible promoter can be operably linked to a nucleotide sequence encoding a signal sequence which is operably linked to a gene for expression in potatoes. Any inducible promoter can be used (See, Ward, et al., Plant Mol. Biol., 22:361-366, 1993). Exemplary inducible promoters include, but are not limited to, a stress-inducible Arabidopsis rd29A promoter (Pino et al., Plant Biotechnol. J. 2007 September: 5 (5): 591-604) and a light-inducible promoter Lhea3 (Meiyalaghan et al., Euphytica 147 (3), 2006). A constitutive promoter can be operably linked to a gene for expression in potatoes or the constitutive promoter is operably linked to a nucleotide sequence encoding a signal sequence which is operably linked to a gene for expression in potatoes. Exemplary constitutive promoters include, but are not limited to, the promoters from plant viruses such as the 35S promoter from CaMY (Odell, et. al., Nature, 313:810-812 (1985) and the promoters from such genes as rice actin (McElroy, et al., Plant Cell, 2:163-171 (1990)); ubiquitin (Christensen, et al., Plant Mol. Biol., 12:619-632 (1989); Christensen, et al., Plant Mol. Biol., 18:675-689 (1992)); pEMU (Last, et al., Theor. Appl. Genet., 81:581-588 (1991)); MAS (Velten, et al., EMBO J., 3:2723-2730 (1984)); and maize H3 histone (Lepetit, et al., Mol. Gen. Genetics, 231:276-285 (1992); Atanassova, et al., Plant Journal, 2 (3): 291-300 (1992)). The ALS promoter, XbaI/NcoI fragment 5′ to the Brassica napus ALS3 structural gene (or a nucleotide sequence similarity to said XbaI/NcoI fragment), is another exemplary constitutive promoter. See also, U.S. Pat. No. 5,659,026. A tissue-specific promoter can be operably linked to a gene for expression in potato. Optionally, the tissue-specific promoter is operably linked to a nucleotide sequence encoding a signal sequence which is operably linked to a gene for expression in potato. Plants transformed with a gene of interest operably linked to a tissue-specific promoter produce the protein product of the transgene exclusively, or preferentially, in a specific tissue. Exemplary tissue-specific or tissue-preferred promoters include, but are not limited to the C(4)-PEPC promoter, see Ghasimi et al., Plant Cell Rep. 2009 (12): 1869-79; and Lim et al., Mol Cells. 2012; 34 (1): 53-59. Transport of a protein produced by transgenes to a subcellular compartment, such as the chloroplast, vacuole, peroxisome, glyoxysome, cell wall, or mitochondrion, or for secretion into the apoplast, can be accomplished by means of operably linking the nucleotide sequence encoding a signal sequence to the 5′ and/or 3′ region of a gene encoding the protein of interest. Targeting sequences at the 5′ and/or 3′ end of the structural gene may determine during protein synthesis and processing where the encoded protein is ultimately compartmentalized. The presence of a signal sequence directs a polypeptide to either an intracellular organelle or subcellular compartment or for secretion to the apoplast. Many signal sequences are well-known in the art. See, for example, Becker, et al., Plant Mol. Biol., 20:49 (1992); Knox, C., et al., Plant Mol. Biol., 9:3-17 (1987); Lerner, et al., Plant Physiol., 91: 124-129 (1989); Frontes, et al., Plant Cell, 3:483-496 (1991); Matsuoka, et al., Proc. Natl. Acad. Sci., 88:834 (1991): Gould, et al., J. Cell. Biol., 108:1657 (1989); Creissen, et al., Plant 1, 2:129 (1991); Kalderon, et al., Cell, 39:499-509 (1984); Steifel, et al., Plant Cell, 2:785-793 (1990).

With transgenic plants, according to one example, a foreign protein can be produced in commercial quantities. Thus, techniques for the selection and propagation of transformed plants, yield a plurality of transgenic plants which are harvested in a conventional manner, and a foreign protein can then be extracted from a tissue of interest or from total biomass. Protein extraction from plant biomass can be accomplished by known methods (e.g., see Heney and Orr, Anal. Biochem., 114:92-6, 1981). In one example, the transgenic plant provided for commercial production of foreign protein is a potato plant. In another embodiment, the biomass of interest is potato tubers and potato seed. For the relatively small number of transgenic plants that show higher levels of expression, a genetic map can be generated, for example using RFLP, PCR, and SSR analysis, which identifies the approximate chromosomal location of the integrated DNA molecule (e.g., see Glick and Thompson, Methods in Plant Molecular Biology and Biotechnology, CRC Press, Inc., Boca Raton, 269:284, 1993). Map information concerning chromosomal location is useful for proprietary protection of a subject transgenic plant. Plants can be genetically engineered to express various phenotypes of agronomic interest. Through the transformation of potato, the expression of genes can be altered to enhance disease resistance, insect resistance, herbicide resistance, agronomie, tuber quality, and other traits. Transformation can also be used to insert DNA sequences which control or help control male-sterility. DNA sequences native to potatoes, as well as non-native DNA sequences, can be transformed into potatoes and used to alter levels of native or non-native proteins. Various promoters, targeting sequences, enhancing sequences, and other DNA sequences can be inserted into the genome for the purpose of altering the expression of proteins. The interruption or suppression of the expression of a gene at the level of transcription or translation (also known as gene silencing or gene suppression) can be desirable for genetic engineering in plants.

Techniques for gene silencing include, but are not limited to, knock-outs (such as by insertion of a transposable element such as Mu (Vicki Chandler, The Maize Handbook, Ch. 118 (Springer-Verlag 1994)) or other genetic elements such as a FRT, Lox, or other site specific integration sites; antisense technology (see. e.g., Sheehy, et al., PNAS USA, 85:8805-8809 (1988) and U.S. Pat. Nos. 5,107,065, 5,453,566, and 5,759,829); co-suppression (e.g., Taylor, Plant Cell, 9:1245 (1997); Jorgensen, Trends Biotech., 8 (12): 340-344 (1990); Flavell, PNAS USA, 91:3490-3496 (1994); Finnegan, et al., Bio/Technology, 12:883-888 (1994); Neuhuber, et al., Mol. Gen. Genet., 244:230-241 (1994)); RNA interference (Napoli, et al., Plant Cell, 2:279-289 (1990); U.S. Pat. No. 5,034,323; Sharp, Genes Dev., 13:139-141 (1999); Zamore, et al., Cell, 101:25-33 (2000): Montgomery, et al., PNAS USA, 95:15502-15507 (1998)), virus-induced gene silencing (Burton, et al., Plant Cell. 12:691-705 (2000): Baulcombe, Curr. Op. Plant Bio., 2:109-113 (1999)); target-RNA-specific ribozymes (Haseloff, et al., Nature, 334:585-591 (1988)); hairpin structures (Smith, et al., Nature, 407:319.320 (2000); U.S. Pat. Nos. 6,423,885, 7,138,565, 6,753,139, and 7,713,715); MicroRNA (Aukerman & Sakai, Plant Cell, 15:2730-2741 (2003)); ribozymes (Steinecke, et al., EMBO J., 11:1525 (1992); Perriman, et al., Antisense Res. Dev., 3:253 (1993)); oligonucleotide mediated targeted modification (e.g., U.S. Pat. Nos. 6,528,700 and 6,911,575); Zn-finger targeted molecules (e.g., U.S. Pat. Nos. 7,151,201, 6,453,242, 6,785,613, 7,177,766 and 7,788,044); and other methods or combinations of the above methods.

The foregoing methods for transformation can be to produce a transgenic potato variety. The transgenic variety can be crossed with another (non-transformed or transformed) variety to produce a new transgenic variety. Alternatively, a genetic trait that has been engineered into a particular potato line using the foregoing transformation techniques could be moved into another line using traditional backcrossing techniques. For example, a backcrossing approach could be used to move an engineered trait from a public, non-elite variety into an elite variety, or from a variety containing a foreign gene in its genome into a variety or varieties that do not contain that gene. As used herein, “crossing” can refer to a simple x by y cross or the process of backcrossing depending on the context. In one example, agronomic genes can be expressed in transformed plants, such as phenotypes of agronomic interest. Exemplary genes include, but are not limited to, those categorized below.

### Male Sterility

The inbred and hybrid potato plants provided herein can include male sterility. Several methods of conferring genetic male sterility in potatoes are known. Male sterility occurs more often in tetraploid cultivars and related taxa (Grun, et al., Genetics 47:1321-1333 (1962)). The male sterility is a consequence of nuclear-cytoplasm interactions, where the dominant Ms gene interacts with the cytoplasm of S. tuberosum to cause male sterility and the dominant Rt gene restores fertility (Iwanaga, et al., Am. Potato J. 68:19-28, 1991).

### Herbicide Resistance

The inbred and hybrid potato plants provided herein can be made herbicide resistant, for example by introducing one or more herbicide genes provided herein into potato cultivar ‘UNR-01’, for example using transformation and crossing. Numerous herbicide resistance genes are known and can be used with the methods and plants provided herein. In particular examples, a herbicide resistance gene confers tolerance to an herbicide comprising glyphosate, sulfonylurea, imidazalinone, dicamba, glufosinate, phenoxy proprionic acid, cyclohexone, triazine, benzonitrile, broxynil, L-phosphinothricin, cyclohexanedione, chlorophenoxy acetic acid, or combinations thereof.

In one example the herbicide resistance gene is a gene that confers resistance to an herbicide that inhibits the growing point or meristem, such as an imidazalinone or a sulfonylurea. Exemplary genes in this category code for mutant ALS and AHAS enzyme as described, for example, by Lee et al. (1988. Embryo J. 7:1241-8) and Miki et al. (1990. Theoret. Appl. Genet. 80:449-458). In one non-limiting example, the herbicide resistance gene is a gene that confers resistance to the sulfonylurea herbicide nicosulfuron.

Resistance genes for glyphosate (e.g., resistance conferred by mutant 5-enolpyruvl-3 phosphikimate synthase (EPSPS) enzyme and aroA genes) and other phosphono compounds such as glufosinate (phosphinothricin acetyl transferase (PAT) and Streptomyces hygroscopicus phosphinothricin-acetyl transferase (bar) genes) can be used (e.g., see U.S. Pat. No. 4,940,835). Examples of specific EPSP transformation events conferring glyphosate resistance are described, for example, in U.S. Pat. Nos. 6,040,497 and 7,632,985. The MON89788 event disclosed in U.S. Pat. No. 7,632,985 can be used to confer glyphosate tolerance in combination with an increase in average yield relative to prior events. Exemplary PAT sequences are provided in RE44962.

DNA molecules encoding a mutant aroA gene are known (e.g., ATCC accession number 39256 and U.S. Pat. No. 4,769,061), as are sequences for glutamine synthetase genes, which confer resistance to herbicides such as L-phosphinothricin (e.g., U.S. Pat. No. 4,975,374), phosphinothricin-acetyltransferase (e.g., U.S. Pat. No. 5,879,903). DeGree F. et al. (1989. Bio/Technology 61-64) describe the production of transgenic plants that express chimeric bar genes coding for phosphinothricin acetyl transferase activity. Exemplary genes conferring resistance to phenoxy propionic acids and cyclohexones, such as sethoxydim and haloxyfop are the Acct-S1, Accl-S2 and Acct-S3 genes described by Marshall et al. (1992. Theor Appl Genet. 83:435-442).

Glyphosate resistance can be imparted to plants by expressing a gene that encodes a glyphosate oxido-reductase enzyme (see U.S. Pat. Nos. 5,776,760 and 5,463,175, incorporated herein by reference in their entireties). In addition, glyphosate resistance can be imparted to plants by the over expression of genes encoding glyphosate N-acetyltransferase. See, for example, U.S. Pat. No. 7,462,481. A DNA molecule encoding a mutant aroA gene can be obtained under ATCC Accession No. 39256, and the nucleotide sequence of the mutant gene is disclosed in U.S. Pat. No. 4,769,061. European Patent Appl. No. 0333033 and U.S. Pat. No. 4,975,374 disclose nucleotide sequences of glutamine synthetase genes which confer resistance to herbicides such as L-phosphinothricin. The PAT gene sequence is provided in European Patent No. 0242246 to Leemans, et al. DeGree F., et al., Bio/Technology, 7:61 (1989) describe the production of transgenic plants that express chimeric bar genes coding for phosphinothricin acetyl transferase activity. Exemplary of genes conferring resistance to phenoxy proprionic acids and cyclohexones, such as sethoxydim and haloxyfop are the Acc1-S1, Acc1-S2, and Acc2-S3 genes described by Marshall, et al., Theor. Appl. Genet., 83:435 (1992).

Genes conferring resistance to an herbicide that inhibits photosynthesis can also be used, such as, a triazine (psbA and gs+ genes) and a benzonitrile (nitrilase gene) (see Przibilla et al., 1991. Plant Cell. 3:169-174). Nucleotide sequences for nitrilase genes are disclosed in U.S. Pat. No. 4,810,648, and DNA molecules containing these genes are available under ATCC Accession Nos. 53435, 67441, and 67442. Cloning and expression of DNA coding for a glutathione S-transferase is described by Hayes et al. (1992. Biochem. J. 285:173).

U.S. Patent Publication No: 20030135879 describes dicamba monooxygenase (DMO) from Pseuodmonas maltophilia, which is involved in the conversion of an herbicidal form of the herbicide dicamba to a non-toxic 3,6-dichlorosalicylic acid and thus can be used for producing plants tolerant to this herbicide.

Protoporphyrinogen oxidase (PPO) is the target of the PPO-inhibitor class of herbicides; a PPO-inhibitor resistant PPO gene was identified in Amaranthus tuberculatus (Patzoldt et al., PNAS. 103 (33): 12329-2334, 2006). The herbicide methyl viologen inhibits CO₂ assimilation. Foyer et al. (Plant Physiol., 109:1047-1057, 1995) describe a plant overexpressing glutathione reductase (OR) which is resistant to methyl viologen treatment. Bromoxynil resistance by introducing a chimeric gene containing the bxn gene (Science, 242 (4877): 419-23, 1988).

4-hydroxyphenylpyruvate dioxygenase (HPPD) is a target of the HPPD-inhibiting herbicides, which deplete plant plastoquinone and vitamin E pools. Rippert, et al. (Plant Physiol., 134:92, 2004) describes an HPPD-inhibitor resistant tobacco plant that was transformed with a yeast-derived prephenate dehydrogenase (PDH) gene. Protoporphyrinogen oxidase (PPO) is the target of the PPO-inhibitor class of herbicides; a PPO-inhibitor resistant PPO gene was recently identified in Amaranthus tuberculatus (Patzoldt, et al., PNAS, 103 (33): 12329, 2006). The herbicide methyl viologen inhibits CO₂ assimilation. Foyer, et al. (Plant Physiol., 109:1047, 1995) describe a plant overexpressing glutathione reductase (GR) that is resistant to methyl viologen treatment.

Siminszky (Phytochemistry Reviews, 5:445, 2006) describes plant cytochrome P450-mediated detoxification of multiple, chemically unrelated classes of herbicides. Modified bacterial genes have been successfully demonstrated to confer resistance to atrazine, an herbicide that binds to the plastoquinone-binding membrane protein QB in photosystem II to inhibit electron transport. For example, Cheung, et al. (PNAS, 85:391, 1988) describe tobacco plants expressing the chloroplast psbA gene from an atrazine-resistant biotype of Amaranthus hybridus fused to the regulatory sequences of a nuclear gene, and Wang, et al. (Plant Biotech. J., 3:475, 2005) describe transgenic alfalfa, Arabidopsis, and tobacco plants expressing the atzA gene from Pseudomonas sp. that were able to detoxify atrazine.

The metabolism of chlorophenoxyacetic acids, such as, for example 2,4-D herbicide, is known. Genes or plasmids that contribute to the metabolism of such compounds are described, for example, by Muller et al. (2006. Appl. Environ. Microbiol. 72 (7): 4853-4861), Don and Pemberton (1981. J Bacteriol 145 (2): 681-686), Don et al. (1985. J Bacteriol 161 (1): 85-90) and Evans et al. (1971. Biochem J 122 (4): 543-551). Bayley, et al. (Theor. Appl. Genet., 83:645, 1992) describe 2,4-D-resistant transgenic tobacco and cotton plants using the 2,4-D monooxygenase gene tfdA from Alcaligenes eutrophus plasmid pJP5. U.S. Patent Application Publication No. 20030135879 describes the isolation of a dicamba monooxygenase (DMO) gene from Pseudomonas maltophilia that is involved in the conversion of dicamba to a non-toxic 3,6-dichlorosalicylic acid and thus may be used for producing plants tolerant to this herbicide.

Acetohydroxy acid synthase, which makes plants that express this enzyme resistant to multiple types of herbicides, has been introduced into a variety of plants. See, Hattori, et al., Mol. Gen. Genet., 246:419 (1995). Other genes that confer tolerance to herbicides include a gene encoding a chimeric protein of rat cytochrome P4507A1 and yeast NADPH-cytochrome P450 oxidoreductase (Shiota, et al., Plant Physiol., 106:17 (1994)); genes for glutathione reductase and superoxide dismutase (Aono, et al., Plant Cell Physiol., 36:1687 (1995)); and genes for various phosphotransferases (Datta, et al., Plant Mol. Biol., 20:619 (1992)).

Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306, 6,282,837, 5,767,373, and 6,084,155.

Other examples of herbicide resistance have been described, for example, in U.S. Pat. Nos. 6,803,501; 6,448,476; 6,248,876; 6,225,114; 6,107,549; 5,866,775; 5,804,425; 5,633,435; 5,463,175.

### Disease Resistance

A gene conferring disease resistance can be introduced into ‘UNR-01’ or progeny thereof, for example by introducing one or more disease resistance genes provided herein into potato cultivar ‘UNR-01’, for example using transformation and crossing. Exemplary pathogens and pests include bacteria, fungi, viruses, and insects.

Plant defenses are often activated by specific interaction between the product of a disease resistance gene (R) in the plant and the product of a corresponding avirulence (Avr) gene in the pathogen. A plant, such as ‘UNR-01’ or progeny thereof, can be transformed with cloned resistance gene to engineer potato plants that are resistant to specific pathogen strains. See, for example Jones et al. (1994. Science 266:789) (tomato Cf-9 gene for resistance to Cladosporium fulvum); Martin et al. (1993. Science 262 (5138): 1432-1436) (tomato Pto gene for resistance to Pseudomonas syringae pv.); and Mindrinos et al. (1994. Cell 78:1089-1099) (Arabidopsis RSP2 gene for resistance to Pseudomonas syringae).

A viral-invasive protein or a complex toxin derived therefrom can be used for viral disease resistance. For example, the accumulation of viral coat proteins in transformed plant cells imparts resistance to viral infection and/or disease development effected by the virus from which the coat protein gene is derived, as well as by related viruses (Beachy et al. 1990. Annu Rev Phytopathol 28:451-474). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus.

A virus-specific antibody can also be used. For example, Tavladoraki et al. (1993. Nature 366:469-472) show that transgenic plants expressing recombinant antibody genes are protected from virus attack.

Additional means of inducing whole-plant resistance to a pathogen include modulation of the systemic acquired resistance (SAR) or pathogenesis related (PR) genes, for example genes homologous to the Arabidopsis thaliana NIM1/NPR1/SAI1, and/or by increasing salicylic acid production.

A developmental-arrestive protein produced in nature by a plant can be introduced into ‘UNR-01’ or progeny thereof. Logemann et al. (1992. Biotechnology, 10:305), for example, disclose transgenic plants expressing a barley ribosome-inactivating gene have an increased resistance to fungal disease. Plant defensins may be used to provide resistance to fungal pathogens (Thomma et al., Planta, 216:193, 2002). Other examples of fungal disease resistance are provided in U.S. Pat. Nos. 6,653,280; 6,573,361; 6,506,962; 6,316,407; 6,215,048; 5,516,671; 5,773,696; 6,121,436; 6,316,407; and 6,506,962.

A gene conferring resistance to a pest, such as the Colorado potato beetle can be introduced into ‘UNR-01’ or progeny thereof (e.g., Mi, et al., C. R. Biol. 2015, 338 (7): 443-50; and the potato tuber moth, Davidson et al., J. Amer. Soc. Hort. Sci. 127 (4): 590-596).

A Bacillus thuringiensis (Bt) protein (a Cry toxin), a derivative thereof or a synthetic polypeptide modeled thereon can be introduced into ‘UNR-01’ or progeny thereof (e.g., Geiser et al., 1986. Gene 48:109, discloses a Bt 8-endotoxin gene). Moreover, DNA molecules encoding δ-endotoxin genes can be obtained from the ATCC (Manassas, VA), for example under ATCC Accession Nos. 40098, 67136, 31995 and 31998. In one example, the Bacillus thuringiensis (Bt) protein is a member of the Cry1 class, and is active primarily against larval stages of the order Lepidoptera. Examples include Cry1Ab (Bt11), Cry1Ac, and Cry1F (e.g., Cry1Fa2 (TC1507)), as well as variants and truncations thereof that provide insect resistance. In one example, the Bacillus thuringiensis (Bt) protein is a member of the Cry2 class or the Cy3 class (such as Cy34Ab1, Cry35ab1).

In one example lectin is introduced into ‘UNR-01’ or progeny thereof. For example, Van Damme et al. (1994. Plant Mol Biol 24 (5): 825-830) disclose several Clivia miniata mannose-binding lectin genes.

A vitamin-binding protein can also be introduced into ‘UNR-01’ or progeny thereof, such as avidin. See WO 1994/000992, which teaches the use of avidin and avidin homologues as larvicides against insect pests.

An enzyme inhibitor, for example, a protease or proteinase inhibitor or an amylase inhibitor can be introduced into ‘UNR-01’ or progeny thereof. For example, Abe et al. (1987. J. Biol. Chem. 262:16793-7) disclose a rice cysteine proteinase inhibitor, Genbank Accession Nos. Z99173.1 and DQ009797.1 disclose proteinase inhibitor coding sequences, Huub et al. (Plant Molec. Biol., 21:985, 1993) describes the nucleotide sequence of cDNA encoding tobacco proteinase inhibitor I, and Sumitani et al. (1993. Plant Mol. Biol. 21:985) discloses the nucleotide sequence of Streptomyces nitrosporeus α-amylase inhibitor.

An insect-specific hormone or pheromone, such as an ecdysteroid or juvenile hormone, a variant thereof, a mimetic based thereon, or an antagonist or agonist thereof, can be introduced into ‘UNR-01’ or progeny thereof. For example, Hammock et al. (1990. Nature 344:458-461) disclose juvenile hormone esterase, an inactivator of juvenile hormone. Further, Gade and Goldsworthy (Eds., Physiological Systems in Insects, Elsevier Academic Press, Burlington, Mass., 2007) describe allostatins and their potential use in pest control, and Palli et al. (Vitam. Horm., 73:59, 2005) describes the use of ecdysteroid and ecdysteroid receptor in agriculture. Additionally, Price et al., (Insect Mol. Biol., 13:469, 2004) identified the diuretic hormone receptor (DHR) as a candidate target of insecticides.

An insect-specific peptide or neuropeptide which, upon expression in ‘UNR-01’ or progeny thereof, disrupts the physiology of the affected pest. For example, see Regan, J. Biol. Chem., 269:9 (1994) (expression cloning yields DNA coding for insect diuretic hormone receptor); Pratt, et al., Biochem. Biophys. Res. Comm., 163:1243 (1989) (an allostatin is identified in Diploptera puntata); Chattopadhyay, et al., Critical Reviews in Microbiology, 30 (1): 33-54 (2004); Zjawiony, J. Nat. Prod., 67 (2): 300-310 (2004); Carlini & Grossi-de-Sa, Toxicon, 40 (11): 1515-1539 (2002); Ussuf, et al., Curr Sci., 80 (7): 847-853 (2001); Vasconcelos & Oliveira, Toxicon, 44 (4): 385-403 (2004). See also, U.S. Pat. No. 5,266,317 which discloses genes encoding insect-specific, paralytic neurotoxins.

An insect-specific venom produced in nature by a snake, a wasp, etc. can be introduced into ‘UNR-01’ or progeny thereof. For example, see, Pang, et al., Gene, 116:165 (1992), for disclosure of heterologous expression in plants of a gene coding for a scorpion insectotoxic peptide.

An enzyme responsible for hyperaccumulation of a monoterpene, a sesquiterpene, a steroid, hydroxamic acid, a phenylpropanoid derivative, or another non-protein molecule with insecticidal activity can be introduced into ‘UNR-01’ or progeny thereof.

An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase, and a glucanase, whether natural or synthetic, can be introduced into ‘UNR-01’ or progeny thereof. See, U.S. Pat. No. 5,955,653 which discloses the nucleotide sequence of a callase gene. DNA molecules which contain chitinase-encoding sequences can be obtained, for example, from the ATCC under Accession Nos. 39637 and 67152. See also, Kramer, et al., Insect Biochem. Molec. Biol., 23:691 (1993), who teach the nucleotide sequence of a cDNA encoding tobacco hornworm chitinase, and Kawalleck, et al., Plant Molec. Biol., 21:673 (1993), who provide the nucleotide sequence of the parsley ubi4-2 polyubiquitin gene, U.S. Pat. Nos. 7,145,060, 7,087,810, and 6,563,020.

A hydrophobic moment peptide can be introduced into ‘UNR-01’ or progeny thereof (e.g., U.S. Pat. No. 5,580,852, which discloses peptide derivatives of tachyplesin which inhibit fungal plant pathogens, and U.S. Pat. No. 5,607,914 which teaches synthetic antimicrobial peptides that confer disease resistance).

A membrane permease, a channel former or a channel blocker can be introduced into ‘UNR-01’ or progeny thereof (e.g., see Jaynes, et al., Plant Sci, 89:43 (1993), of heterologous expression of a cecropin-13 lytic peptide analog to render transgenic tobacco plants resistant to Pseudomonas solanacearum).

An insect-specific antibody or an immunotoxin derived therefrom can be introduced into ‘UNR-01’ or progeny thereof. Thus, an antibody targeted to a critical metabolic function in the insect gut inactivates an affected enzyme, killing the insect.

A developmental-arrestive protein produced in nature by a pathogen or a parasite can be introduced into ‘UNR-01’ or progeny thereof. Thus, fungal endo-α-1,4-D-polygalacturonases facilitate fungal colonization and plant nutrient release by solubilizing plant cell wall homo-α-1,4-D-galacturonase. See, Lamb, et al., Bio/Technology, 10:1436 (1992). The cloning and characterization of a gene which encodes a bean endopolygalacturonase-inhibiting protein is described by Toubart, et al., Plant 1, 2:367 (1992).

Genes involved in the Systemic Acquired Resistance (SAR) Response and/or the pathogenesis-related genes can be introduced into ‘UNR-01’ or progeny thereof (e.g., see Briggs, Current Biology, 5 (2) (1995); Pieterse & Van Loon, Curr. Opin. Plant Bio., 7 (4): 456-64 (2004); and Somssich, Cell, 113 (7): 815-6 (2003)).

Antifungal genes can be introduced into ‘UNR-01’ or progeny thereof (e.g., see, Cornelissen and Melchers, Plant Physiol., 101:709-712 (1993); Parijs, et al., Planta, 183:258-264 (1991); and Bushnell, et al., Can. J of Plant Path., 20 (2): 137-149 (1998); U.S. Pat. No. 6,875,907).

Detoxification genes, such as for fumonisin, beauvericin, moniliformin, and zearalenone and their structurally-related derivatives can be introduced into ‘UNR-01’ or progeny thereof (e.g., see U.S. Pat. No. 5,792,931).

Cystatin and cysteine proteinase inhibitors can be introduced into ‘UNR-01’ or progeny thereof (e.g., see U.S. Pat. No. 7,205,453).

Defensin genes can be introduced into ‘UNR-01’ or progeny thereof (e.g., see U.S. Pat. Nos. 6,911,577, 7,855,327, 7,855,328, 7,897,847, 7,910,806, 7,919,686, and 8,026,415).

Genes conferring resistance to nematodes can be introduced into ‘UNR-01’ or progeny thereof (e.g., see U.S. Pat. Nos. 5,994,627, 6,228,992, and 6,294,712; Urwin et al., Planta, 204:472-479 (1998); Williamson, Curr Opin Plant Bio., 2 (4): 327-31 (1999)).

Genes conferring resistance to potato late blight, such as Rpi-Vnt1 can be introduced into ‘UNR-01’ or progeny thereof.

Genes conferring resistance to potato leaf roll virus (PLRV) through gene silencing mechanism, such as plrv orf1 and 2, can be introduced into ‘UNR-01’ or progeny thereof.

Genes conferring resistance to potato virus Y (PVY) through “pathogen-derived resistance” mechanism, such as pvy cp, can be introduced into ‘UNR-01’ or progeny thereof (e.g., see Song, Dissertation, Technical University of Munchen, dated Jul. 26, 2004).

An insect-specific antibody or an immunotoxin derived therefrom or a developmental-arrestive protein can be introduced into ‘UNR-01’ or progeny thereof. For example, Taylor et al. (1994. Seventh Intl. Symposium on Molecular Plant-Microbe Interactions (Edinburgh Scotland), Abstract #497) describe enzymatic inactivation in transgenic tobacco via production of single-chain antibody fragments.

Bacterial disease resistance can be introduced into ‘UNR-01’ or progeny thereof, for example as described, for example, in U.S. Pat. No. 5,516,671.

Any of the above-listed disease or pest resistance genes can be introduced into the ‘UNR-01’ through a variety of means including, but not limited to, transformation and crossing.

### Value Added Traits

Genes conferring modified fatty acid metabolism can be introduced into ‘UNR-01’ and its progeny, such as an antisense nucleic acid of stearoyl acyl carrier protein (ACP) desaturase genes (EC 1.14.99.6) (e.g., Knutzon et al. 1992. PNAS 89:2624-2628). Fatty acid desaturases can be introduced into ‘UNR-01’ and its progeny, such as Saccharomyces cerevisiae OLE1 gene encoding 49-fatty acid desaturase, an enzyme which forms the monounsaturated palmitoleic (16:1) and oleic (18:1) fatty acids from palmitoyl (16:0) or stearoyl (18:0) CoA (McDonough et al., 1992. J Biol Chem 267 (9): 5931-5936); a gene encoding a stearoyl-acyl carrier protein-9 desaturase from castor (Fox et al. 1993. PNAS 90 (6): 2486-2490); Δ6- and Δ12-desaturases from the cyanobacteria Synechocystis responsible for the conversion of linoleic acid (18:2) to gamma-linolenic acid (18:3 gamma) (Reddy et al., 1993. Plant Mol Biol 22 (2): 293-300); a gene from Arabidopsis thaliana that encodes an omega-3 desaturase (Arondel et al. 1992. Science 258:1353-5); plant Δ9-desaturases (WIPO Publication No. WO 1991/013972) and corn and Brassica Δ15 desaturases (European Patent Application Publ. No. EP 0616644).

Phytate metabolism can also be modified in ‘UNR-01’ and its progeny by introduction of a phytase-encoding gene to enhance breakdown of phytate, adding more free phosphate to the transformed plant (e.g., see Van Hartingsveldt et al., Gene 127:87-94, 1993, for an Aspergillus niger phytase gene) or up-regulation of a gene that reduces phytate content.

A number of genes can be used to alter carbohydrate metabolism in ‘UNR-01’ and its progeny. For example, plants can be transformed with a gene coding for an enzyme that alters the branching pattern of starch, or a gene altering thioredoxin, such as NTR and/or TRX (See, U.S. Pat. No. 6,531,648), and/or a gamma zein knock out or mutant, such as es27 or TUSC27 or en27 (See. U.S. Pat. Nos. 6,858.778, 7,741,533 and U.S. Publ. No. 2005/0160488). Sec, Shiroza, et al., J. Bacteriol., 170:810 (1988) (nucleotide sequence of Streptococcus mutans fructosyltransferase gene); Steinmetz, et al., Mol. Gen. Genet., 200:220 (1985) (nucleotide sequence of Bacillus subtilis levansucrase gene); Pen, et al., Bio/Technology, 10:292 (1992) (production of transgenic plants that express Bacillus licheniformis α-amylase); Elliot, et al., Plant Mol. Biol., 21:515 (1993) (nucleotide sequences of tomato invertase genes); Søgaard, et al., J. Biol. Chem., 268:22480-22484 (1993) (site-directed mutagenesis of barley α-amylase gene); Fisher, et al., Plant Physiol., 102:1045 (1993) (maize endosperm starch branching enzyme II); International Pub. No. WO 99/10498 (improved digestibility and/or starch extraction through modification of UDP-D-xylose 4-epimerase, Fragile 1 and 2. Ref 1, HCHL, C4H); U.S. Pat. No. 6,232,529 (method of producing high oil seed by modification of starch levels (AGP)). The fatty acid modification genes mentioned above may also be used to affect starch content and/or composition through the interrelationship of the starch and oil pathways.

Genes conferring elevated oleic acid via FAD-2 gene modification and/or decreased linolenic acid via FAD-3 can be introduced into ‘UNR-01’ and its progeny, (e.g., see, U.S. Pat. Nos. 5.952.544, 6.063.947, and 6,323,392).

Conjugated linolenic or linoleic acid content, can be altered in ‘UNR-01’ and its progeny, (e.g., see. U.S. Pat. No. 6,593,514. Altering LECI. AGP, Dek1, Superall, milps, and various Ipa genes, such as Ipa1, Ipa3, hpt, or hggt. See, for example, U.S. Pat. Nos. 7,122,658, 7,342,418, 6,232,529, 7,888,560, 6,423,886, 6,197,561, 6,825,397 and 7,157,621; U.S. Publ. No. 2003/0079247; International Publ. No. WO 2003/011015; and Rivera-Madrid, R., et al., Proc. Natl. Acad. Sci., 92:5620-5624 (1995)).

Antioxidant content or composition can be altered in ‘UNR-01’ and its progeny, such as alteration of tocopherol or tocotrienols. See, for example. U.S. Pat. Nos. 6,787,683, 7,154,029 and International Publ. No. WO 00/68393 (involving the manipulation of antioxidant levels through alteration of a phytl prenyl transferase (ppt)); and U.S. Pat. Nos. 7,154,029 and 7.622.658 (through alteration of a homogentisate geranyl geranyl transferase (hggt)).

Essential seed amino acids can be altered in ‘UNR-01’ and its progeny, see, for example, U.S. Pat. No. 6,127,600 (method of increasing accumulation of essential amino acids in seeds); U.S. Pat. No. 6,080,913 (binary methods of increasing accumulation of essential amino acids in seeds); U.S. Pat. No. 5,990,389 and International Publ. No. WO 95/15392 (high lysine); U.S. Pat. No. 5,850,016 (alteration of amino acid compositions in seeds); U.S. Pat. No. 5,885,802 (high methionine); U.S. Pat. No. 5,885,801 and International Publ. No. WO96/01905 (high threonine); U.S. Pat. Nos. 6,664,445, 7,022,895, 7,368,633, and 7,439,420 (plant amino acid biosynthetic enzymes); U.S. Pat. No. 6,459,019 and U.S. application Ser. No. 09/381,485 (increased lysine and threonine); U.S. Pat. No. 6,441,274 (plant tryptophan synthase beta subunit); U.S. Pat. No. 6,346,403 (methionine metabolic enzymes); U.S. Pat. No. 5,939,599 (high sulfur): U.S. Pat. No. 5,912,414 (increased methionine); U.S. Pat. No. 5,633,436 (increasing sulfur amino acid content); U.S. Pat. No. 5,559,223 (synthetic storage proteins with defined structure containing programmable levels of essential amino acids for improvement of the nutritional value of plants); U.S. Pat. No. 6,194,638 (hemicellulosc); U.S. Pat. No. 7,098,381 (UDPGdH); U.S. Pat. No. 6,194,638 (RGP); U.S. Pat. Nos. 6.399.859, 6,930,225, 7,179,955, 6,803,498, 5,850,016, and 7,053,282 (alteration of amino acid compositions in seeds); WO 99/29882 (methods for altering amino acid content of proteins): U.S. application Ser. No. 09/297,418 (proteins with enhanced levels of essential amino acids): WO 98/45458 (engineered seed protein having higher percentage of essential amino acids); WO 01/79516; and U.S. Pat. Nos. 6,803,498, 6,930,225, 7,307, 149, 7,524,933, 7,579,443, 7.838.632, 7.851.597, and 7.982.009 (maize cellulose synthases).

### Resistance to Abiotic Stress

Abiotic stress tolerance in ‘UNR-01’ or in progeny of ‘UNR-01’ can include, but is not limited to, tolerance to stress induced by, for example, flowering, enhancement of nitrogen utilization efficiency, altered nitrogen responsiveness, drought resistance or tolerance, cold resistance or tolerance, heat resistance or tolerance, low or high soil pH level resistance or tolerance, submergence tolerance, tolerance of exposure to heavy metals, oxidative stress tolerance, and salt resistance or tolerance. Such abiotic stress tolerance can increase yield under stress. The inbred and hybrid potato plants provided herein can be made resistant to abiotic stress, for example by introducing one or more genes provided herein into potato cultivar ‘UNR-01’, for example using transformation and crossing.

For example, see U.S. Pat. No. 6,653,535 where water use efficiency is altered through alteration of malate; U.S. Pat. Nos. 5,892,009, 5,965,705, 5,929,305, 5,891,859, 6,417,428, 6,664,446, 6,706,866, 6,717,034, 6,801,104, 6,946,586, 7,238,860, 7,635,800, 7,135,616, 7,193,129, and 7,601,893; and International Publ. Nos. WO 2001/026459, WO 2001/035725, WO 2001/035727, WO 2001/036444, WO 2001/036597, WO 2001/036598, WO 2002/015675, and WO 2002/077185, describing genes, including CBF genes and transcription factors effective in mitigating the negative effects of freezing, high salinity, and drought on plants, as well as conferring other positive effects on plant phenotype; U.S. Publ. No. 2004/0148654, where abscisic acid is altered in plants resulting in improved plant phenotype, such as increased yield and/or increased tolerance to abiotic stress; U.S. Pat. Nos. 6,992,237, 6,429,003, 7,049,115, and 7,262,038, where cytokinin expression is modified resulting in plants with increased stress tolerance, such as drought tolerance, and/or increased yield. See also, WO 02/02776, WO 2003/052063, JP 2002281975, U.S. Pat. No. 6,084,153, WO 01/64898, and U.S. Pat. Nos. 6,177,275 and 6,107,547 (enhancement of nitrogen utilization and altered nitrogen responsiveness). For ethylene alteration, see, U.S. Publ. Nos. 2004/0128719, 2003/0166197, and U.S. application Ser. No. 09/856,834. For plant transcription factors or transcriptional regulators of abiotic stress, see, e.g., U.S. Publ. Nos. 2004/0098764 or 2004/0078852.

Delta-pyrroline-5-carboxylate synthetase (P5CS) from mothbean has been used to provide protection against general osmotic stress. Mannitol-1-phosphate dehydrogenase (mt1D) from E. coli has been used to provide protection against drought and salinity. Choline oxidase (codA from Arthrobactor globiformis) can protect against cold and salt. E. coli choline dehydrogenase (betA) provides protection against salt. Additional protection from cold can be provided by omega-3-fatty acid desaturase (fad7) from Arabidopsis thaliana. Trehalose-6-phosphate synthase and levan sucrase (SacB) from yeast and Bacillus subtilis, respectively, can provide protection against drought (summarized from Annex II Genetic Engineering for Abiotic Stress Tolerance in Plants, Consultative Group On International Agricultural Research Technical Advisory Committee). Overexpression of superoxide dismutase can be used to protect against superoxides, as described in U.S. Pat. No. 5,538,878.

Genes that Create a Site for Site Specific DNA Integration:

Genes that create a site for site-specific DNA integration can be introduced into the disclosed potato variety ‘UNR-01’ or progeny of the disclosed variety. This includes the introduction of FRT sites that may be used in the FLP/FRT system and/or Lox sites that may be used in the Cre/Loxp system. See, for example, Lyznik, et al., Site-Specific Recombination for Genetic Engineering in Plants, Plant Cell Rep, 21:925-932 (2003) and U.S. Pat. No. 6,187,994, which are hereby incorporated by reference. Other systems that may be used include the Gin recombinase of phage Mu (Maeser, et al. (1991); Vicki Chandler, The Maize Handbook, Ch. 118 (Springer-Verlag 1994)); the Pin recombinase of E. coli (Enomoto, et al. (1983)); and the R/RS system of the pSRi plasmid (Araki, et al. (1992)).

### Additional Traits

Additional traits can be introduced into the disclosed potato variety ‘UNR-01’ or progeny of the disclosed variety. Other genes and transcription factors that affect plant growth and agronomic traits, such as yield, flowering, plant growth, and/or plant structure, can be introduced or introgressed into plants. See for example, U.S. Pat. Nos. 6.140,085, and 6,265.637 (CO); U.S. Pat. No. 6,670,526 (ESD4); U.S. Pat. Nos. 6.573.430 and 7,157,279 (TFL): U.S. Pat. No. 6,713,663 (FT); U.S. Pat. Nos. 6,794,560, 6,307,126 (GAI); U.S. Pat. No. 7,045,682 (VRN1); U.S. Pat. Nos. 6,949,694 and 7,253,274 (VRN2); U.S. Pat. No. 6,887,708 (GI); U.S. Pat. No. 7,320,158 (FRI); U.S. Pat. No. 6,307,126 (GAI); U.S. Pat. Nos. 6,762,348 and 7.268.272 (DS and Rht); and U.S. Pat. Nos. 7,345,217, 7,511,190, 7,659,446, and 7,825,296 (transcription factors).

### Tissue Cultures and In Vitro Regeneration of Potato Plants

Tissue cultures of the new potato variety ‘UNR-01’ are provided. Reproduction of the variety can occur by tissue culture and regeneration. A tissue culture includes isolated cells of the same or a different type or a collection of such cells organized into parts of a plant. Exemplary types of tissue cultures include protoplasts, calli, plant clumps, and plant cells that can generate tissue culture that are intact in plants or parts of plants, such as embryos, pollen, flowers, seeds, pods, petioles, leaves, stems, roots, root tips, anthers, pistils, and the like. In a particular example, the tissue culture includes embryos, protoplasts, meristematic cells, pollen, leaves or anthers of the new potato variety ‘UNR-01’. Also provided are potato plants regenerated from such tissue cultures, wherein the regenerated potato plant expresses the physiological and morphological characteristics of the potato variety ‘UNR-01’. Means for preparing and maintaining plant tissue cultures are known (U.S. Pat. Nos. 5,538,880; 5,550,318; 5,959,185, 5,973,234, and 5,977,445).

Tissue culture of various tissues of potatoes and regeneration of plants therefrom is known. See, Ahloowalia, B. S., “Plant regeneration from callus culture in potato” Euphytica. 31 (3): pp 755-759 (December 1982); and Wang, P. J., “Regeneration of Virus-free Potato from Tissue Culture” in Plant Tissue Culture and Its Bio-technological Application, Bartz, et al. (eds). Springer-Verlag Berlin Heidelberg. pp 386-391 (1977). Thus, provided are cells which upon growth and differentiation produce potato plants having the physiological and morphological characteristics of potato variety ‘UNR-01’.

Embryogenic cultures can also be used for regeneration, including regeneration of transgenic plants.

### Commodity Products

Potato variety ‘UNR-01’ has a variety of commodity uses. Fresh potatoes can be cooked (fried, baked, boiled, etc.). Potatoes of potato variety ‘UNR-01’ can be used to make potato chips, frozen potato items such as hash/home fries/French fries, dehydrated potato flakes, potato granules, ingredients in food snacks, potato flour, potato starch, and alcoholic beverages.

Potatoes of potato variety ‘UNR-01’ can be used in non-food uses such as potato starch used by the pharmaceutical, textile, wood, and paper industries as an adhesive, binder, texture agent, and filler, and by oil drilling firms to wash boreholes. Potato starch can also be used in place of polystyrene and other plastics disposable dishes and utensils. Potato peel and other wastes from processing potato variety ‘UNR-01’ can be liquefied and fermented to produce fuel-grade ethanol. Thus, provided are food products or non-food products made from a part of the potato plant variety ‘UNR-01’. The food product may be a French fry, potato chip, dehydrated potato material, potato flakes, or potato granules. In one example the food product is a sliced potato tuber food product. Also provided are t heat-processed tuber product produced from the disclosed potato plant variety ‘UNR-01’, such as a French fry, a potato chip, or a baked potato.

### Example 1

### Materials and Methods

### Plasmid Construct Preparation:

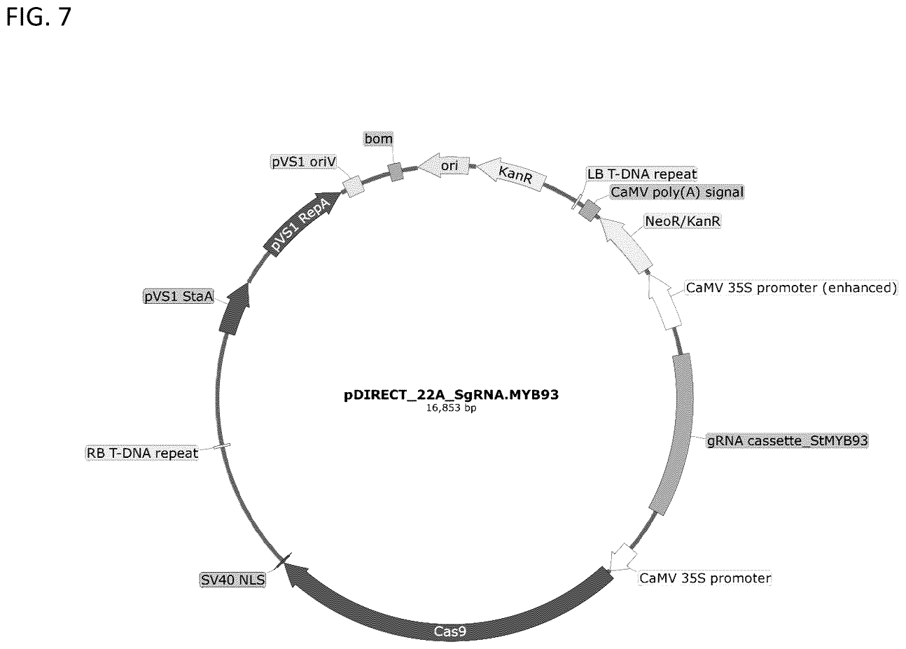

Three gRNAs were designed targeting the StMYB93 (PGSC0003DMG400006408; SEQ ID NO: 1) gene portion at 528 bp, 1546 bp and 1628 bp respectively. One of these gRNAs was positioned after the R2 domain, the other two gRNAs targeted DNA sequences near the 3′ end of the gene (SEQ ID NO: 2). Each of these gRNAs along with a promoter and a terminator were cloned into pDIRECT_22A (FIG. 7). The pDIRECT_22A+gRNA+Cas9 construct was transformed into Agrobacterium tumefaciens GV3101.

### Development of StMYB93 (PGSC0003DMG400006408) Gene-Edited Potatoes:

Leaf explants from in vitro grown potato plants (cv. Atlantic) were co-cultivated with A. tumefaciens GV3101 harboring the pDIRECT_22A+gRNA+Cas9 construct and developed into transgenic potato plants following the regeneration protocol described by (Knapp et al., Molecular and General Genetics MGG, 213 (2): 285-290, 1988; Vulavala et al., Plant Molecular Biology. https://doi.org/10.1007/s11103-017-0619-32017) with slight modification. In brief, leaves were excised from in vitro micropropagated plants and cut into 1 cm pieces. These leaf pieces were co-cultivated with A. tumefaciens GV3101 harboring the pDIRECT_22A+gRNA+Cas9 construct for 2-3 days in dark on basal medium (Murashige and Skoog medium with 30 g/L sucrose and 8 g/L agar, pH 5.8). The leaf explants were further transferred to basal medium supplemented with 5 mg/L α-naphthalene acetic acid (NAA, Duchefa Biochemie V.B., The Netherlands), 0.1 mg/L 6-benzylaminopurine (BAP, Sigma), 500 mg/L claforan (Cefotaxim, Duchefa), and 50 mg/L kanamycin (Sigma). After 2 weeks, the leaves were transferred onto basal medium supplemented with 0.02 mg/L NAA, 2 mg/L zeatin riboside (Duchefa), 0.02 mg/L gibberellic acid (GA3, Duchefa), 500 mg/L claforan and 50 mg/L kanamycin, and incubated under a 16 h photoperiod. They were subcultured every 2 weeks on to fresh media for 6-8 weeks until found shoot regeneration. Emerged shoots were transferred on to rooting media (basal medium supplemented with 500 mg/L cefotaxime and 50 mg/L kanamycin). Genomic DNA was extracted from each plant and the presence of the transgene was confirmed by performing PCR with Neomycin Phosphotransferase II (NPTII) gene-specific primers (Forward Primer: 5′-CACGCAGGTTCTCCGGCCGC-3′, SEQ ID NO: 3; Reverse primer: 5′-TGCGCTGCGAATCGGGAGCG-3′, SEQ ID NO: 4). Confirmed transgenic plants were transferred into potting media in a growth chamber. After acclimatization, the TO plants were transferred to soil filled pots in a greenhouse bay where they were grown to maturity. Tubers were harvested from mature plants and stored at 10° C. for later growth and analysis.

### Molecular Characterization of Gene-Edited Events:

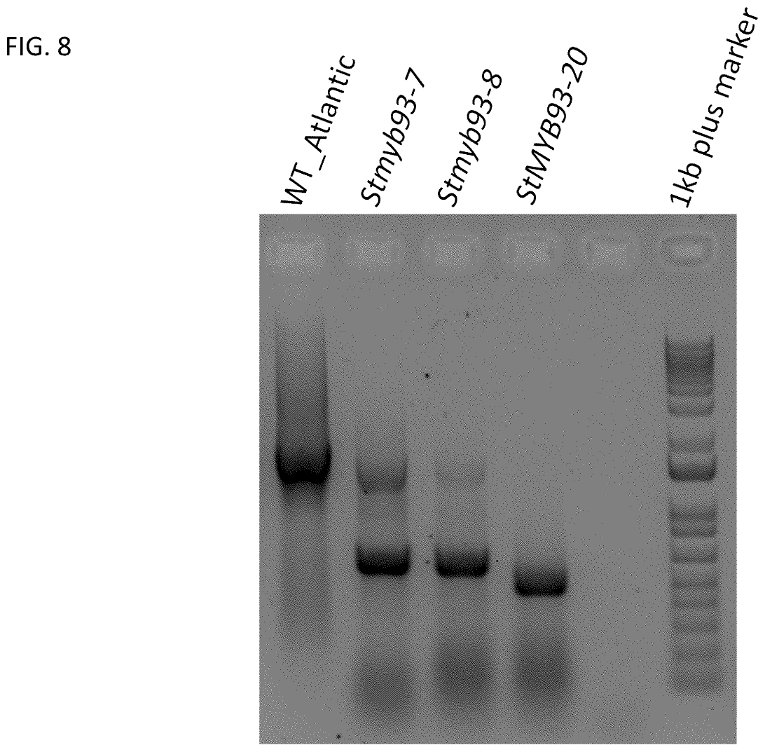

The leaves of transgenic plants growing in the soil in the greenhouse were collected and DNA extracted using the CTAB method as described in (Vulavala et al., 2017). The genomic portion including DNA sequences flanking the edited region of StMYB93 was amplified through PCR using the primers sg. StMYB93_FP (5′-CATGTCTAATTTGGTAGCATC-3′, SEQ ID NO: 5) and sg. StMYB93_RP (5′-GAAATGTCATGCATGAAGTG-3′, SEQ ID NO: 6). A mix of DNA polymerases (Apex taq red mastermix, Cat: 42-138; Platinum SuperFi II DNA polymerase, cat: 12361010) was used to amplify the target DNA sequence per the manufacturer's instructions. Gel electrophoresis demonstrated different sized amplicons indicative of successful gene editing (FIG. 8). Excised individual DNA bands were cloned into pGEMT-easy vector and transformed into Escherichia coli. Sanger sequencing of individual colonies was performed to validate DNA sequences.

### Potato Tuber Storage:

Greenhouse grown potatoes from wildtype and Stmyb93-20 plants were harvested, washed in tap water and dried with paper towels. Tubers were harvested into mesh bags and stored in a cold storage facility at 10-12° C. in the dark. Tubers were stored for more than 14 months. Tuber sprouting was monitored periodically during storage.

### Potato Wound Periderm Suberin Analysis:

After less than seven days of storage, tubers harvested from greenhouse-grown wildtype and Stmyb93-20 plants were subjected to a wound healing experiment to determine wound suberin content and composition. Tubers were cut into halves to induce wounding and stored in an incubator at 12° C. stored over a saturated solution of sodium chloride (NaCl) to maintain a relative humidity of 76% using a randomized complete block design. The developed wound periderm was collected from the wounded surface at 0 minutes, 2d, 7d and 14 days post wounding at a thickness of 3 mm using a razor blade. Samples were snap frozen in liquid nitrogen and stored at −80° C. A cork borer was used to collect samples with a fixed surface area for normalization of suberin data. Suberin content and composition were determined using methods described in (Wahrenburg et al., 2021).

RNA Extraction and qPCR:

Total RNA was extracted using a CTAB method as described in (Vulavala et al., 2017) and purified the RNA using an RNA clean & concentrator kit (Zymo research, cat: R1017) following the manufacturer's instructions. cDNA was synthesized using iScript™ cDNA Synthesis Kit (Bio-Rad, cat: 1708890) with an initial amount of RNA of lug. Synthesized cDNA was diluted 1:40 and 3 ul of diluted cDNA was used as template for qPCR using iTaq™ Universal SYBR® Green Supermix (Bio-Rad, cat: 1725120) following the manufacturer's instructions. Samples were run on a Bio-Rad qPCR machine. Relative expression levels of targeted genes were determined by normalizing to two house-keeping genes StActin (Forward primer-5′-CTAGCAGCATGAAGATTAAGGTG-3′, SEQ ID NO: 7; Reverse primer 5′-GGACAATAGAAGGACCAGATTCG-3′, SEQ ID NO: 8) and StEF1alpha (Forward primer-5′-ATTGGAAACGGATATGCTCCA-3′, SEQ ID NO: 9; Reverse primer 5′-TCCTTACCTGAACGCCTGTCA-3′, SEQ ID NO: 10).

### Reduced Sugar Level Test:

Reduced sugar levels were measured to determine if the Stmyb93-20 tubers were similar to wildtype Atlantic and suitable for frying without producing undesirable dark spots. A commercial kit “Potato Glucose paper strips (Bart ovation, P/N: PSP04B40)” was used following the manufacturer's instructions. Tubers were cut into halves with the test strip applied flat along the length of tuber. Tuber halves were pressed together to ensure that all surfaces of the test strip were in contact with the starchy flesh of the tuber. The test strips were removed, and two minutes were allowed to pass for proper color development on the strip. The color of the test strips was then matched to the color range given with the kit to assess the total reduced sugar levels (glucose).

### Example 2

### Stmyb93-20 Transgenic Event Results in Sprout Suppression

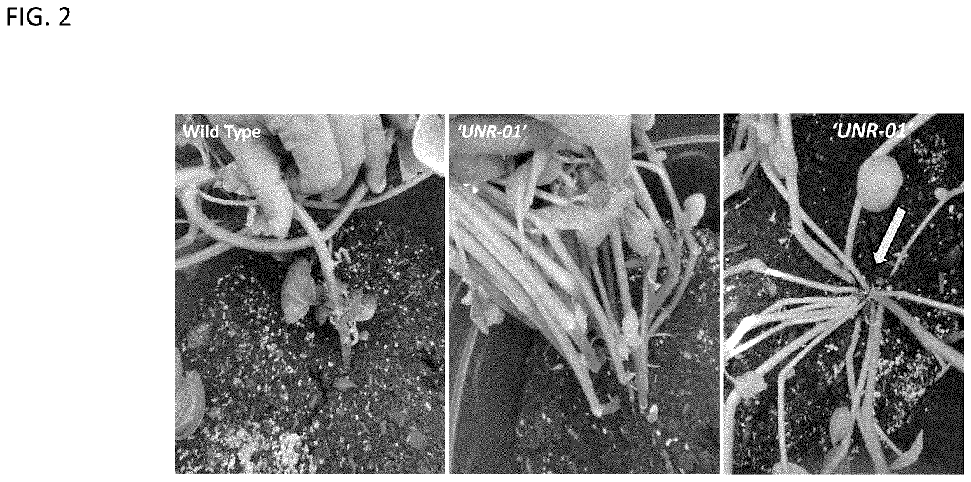

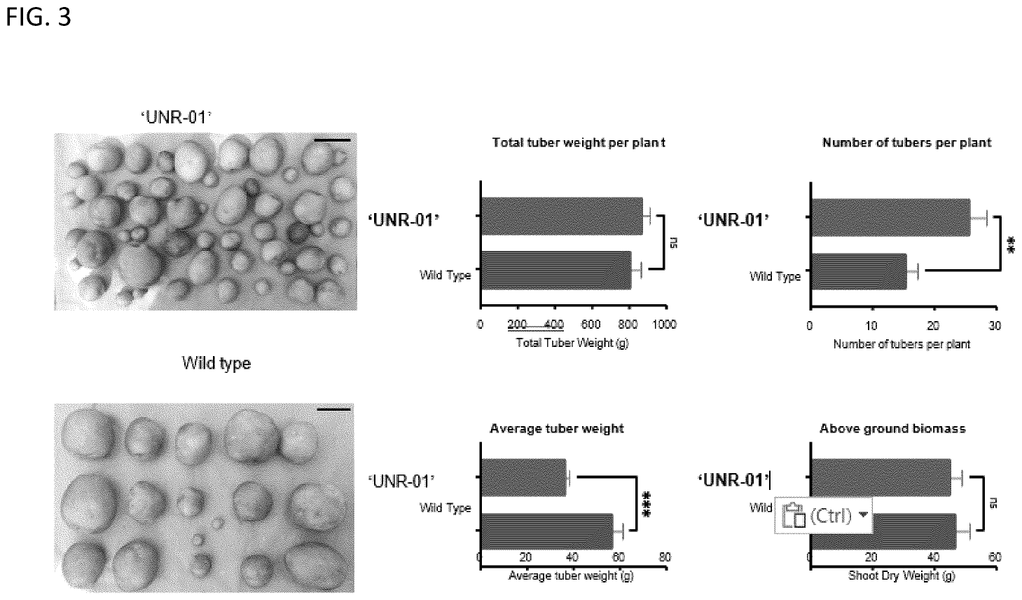

The CRISPR-Cas9 gene-edited potato line exhibited reduced sprouting suppression phenotype even after 10 months of storage. These tubers contained eyes that were either unsprouted or with minimal sprouting when compared with the wildtype which exhibited excessive sprouting (FIGS. 1A-1C). The sprouted eyes from Stmyb93-20 tubers (variety ‘UNR-01’) showed a broken apical dominance resulting in the formation of multiple branches (FIG. 1C). These branches were much less elongated compared to wildtype sprouted stems (FIG. 1A). The stored tubers of ‘UNR-01’ were less desiccated compared to wildtype, as a result the tubers exhibited less shrinkage and maintained a smooth skin texture compared to wildtype (FIGS. 1B & 1C). These phenotypes were further retained for more than 14 months, which is very useful for long-term storage of tubers mitigating the need to apply chemical sprout inhibitors such as Chlorpropham (CIPC), which was recently banned in the United Kingdom and the European Union (https://european-seed.com/2021/04/the-worry-with-cipc/). When tubers from 10 months post storage were grown in soil, ‘UNR-01’ plants emerged with multiple branches as shown in (FIG. 2). ‘UNR-01’ plants yielded the same total weight of tubers as wildtype but yielded a larger number of tubers smaller in size (1-3 cm diameter) than the tubers yielded from wildtype plants in the same growth cycle (FIG. 3). The small sized tubers provide a great opportunity for use as seed tubers or for the edible fresh tuber market thereby being of economic value to the seed and fresh market tuber industry.

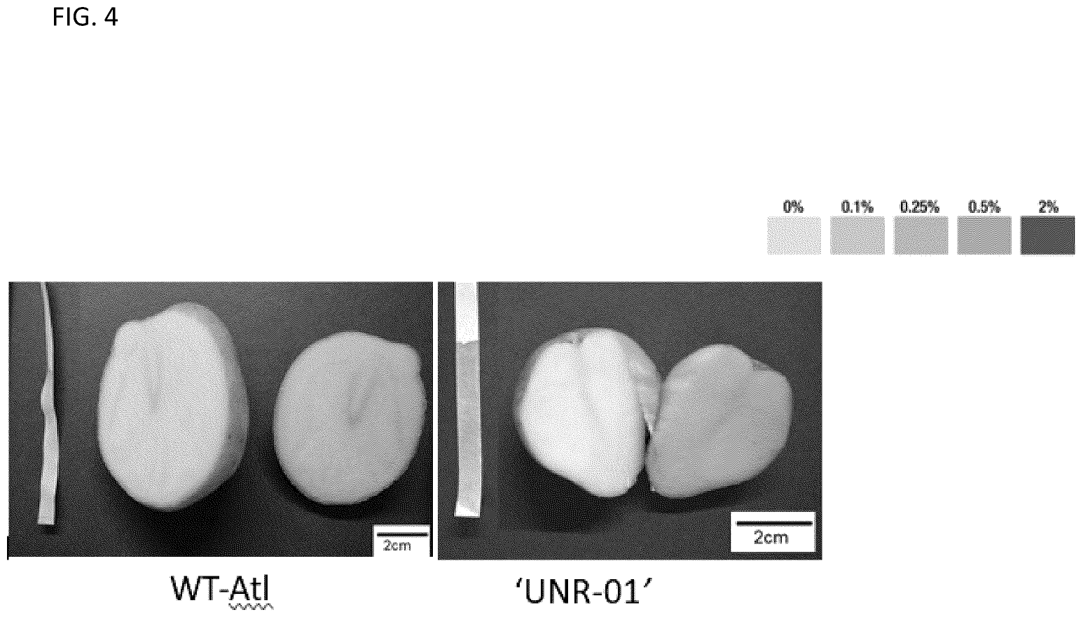

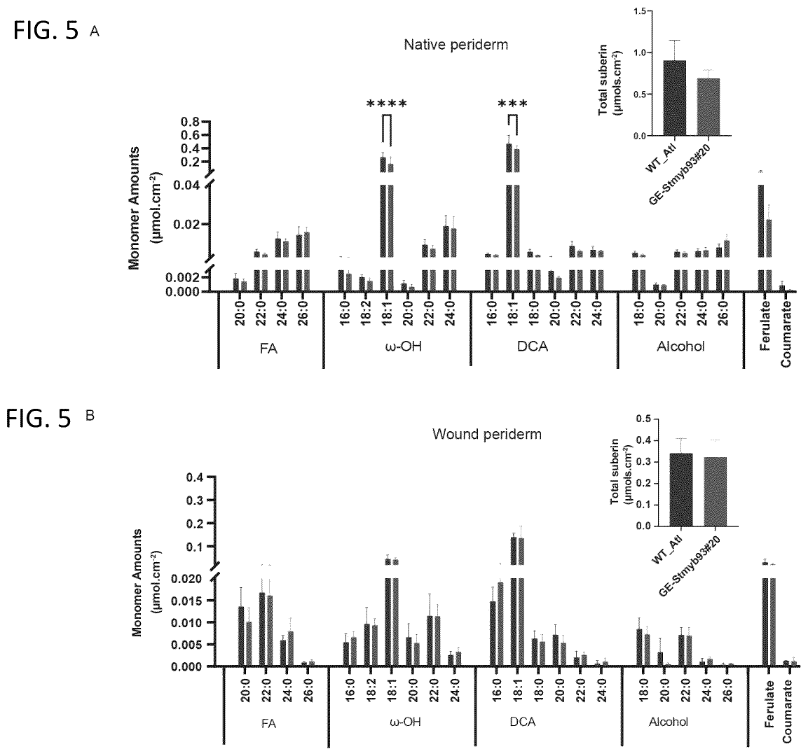

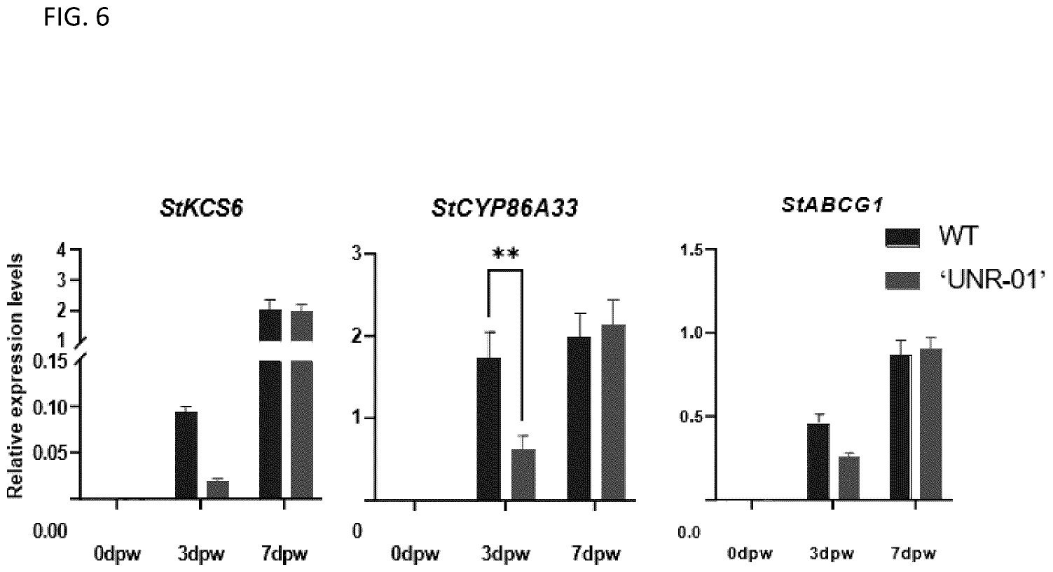

After 10 months in storage, reduced sugar levels in ‘UNR-01’ were equivalent to wildtype tubers (FIG. 4). The ‘UNR-01’ wound healing characteristics were similar to the wild type when a wounding assay was performed (FIG. 5). Two out of three suberin-related genes revealed no change in expression in ‘UNR-01’ during wound periderm development (FIG. 6). This phenotype was consistently observed till >14 months. ‘UNR-01’ potatoes were multiplied, T2 tubers harvested to repeat the storage experiment for reproducibility.

### Example 3

### Allele Specific Editing Patterns were Observed in StMYB93 Event #20

The potatoes cv. Atlantic is an autotetraploid, meaning they contain four copies of the genome. Amplified genomic portion of StMYB93 from wild type and genetic edited (GE) events were run on individual wells of agarose gel electrophoresis (FIG. 8) and found several band sizes in Stmyb93-20 (‘UNR-01’). Sanger sequencing of individual bands showed successful deletion of ˜1.2 kb.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

We claim:

1. A potato tuber, plant, plantlet, plant part, or plant cell of potato variety ‘UNR-01’, wherein a representative potato tuber, plantlet, or tissue culture of the variety has been deposited under Accession No.

2. A potato plant, or a part thereof, produced by growing the potato tuber, plant, plantlet, plant part, or plant cell of claim 1, wherein the plant part comprises at least one cell of potato variety ‘UNR-01’.

3. A potato plant having all of the physiological and morphological characteristics of the plant of claim 2 and comprising a genetically inactivated StMYB93 gene.

4. The potato plant part of claim 2, wherein the plant part is a leaf, pollen, embryo, cotyledon, hypocotyl, meristematic cell, root, root tip, pistil, anther, flowers, ovule, light sprout, petiole, eye, stem, or tuber.

5. A tissue culture produced from the potato plant, or part thereof, of claim 2.

6. A potato plant regenerated from the tissue culture of claim 5, wherein said plant has all of the physiological and morphological characteristics of potato variety ‘UNR-01’.

7. An F₁ potato plant regenerated from the tissue culture of claim 5.

8. A potato light sprout produced by growing the plant of claim 2.

9. A potato plant, or a part thereof, produced by growing the light sprout of claim 8.

10. A potato plant regenerated from tissue culture of the potato plant of claim 9, wherein said potato plant has all of the physiological and morphological characteristics of potato variety ‘UNR-01’.

11. A potato seed produced by growing the potato tuber, or a part of the tuber, of claim 1, wherein the potato seed comprises the genetically inactivated StMYB93 gene that is present in variety ‘UNR-01’.

12. A potato plant, or a part thereof, produced by growing the seed of claim 11.

13. A potato plant regenerated from tissue culture of the potato plant of claim 12, wherein said regenerated plant comprises the genetically inactivated StMYB93 gene that is present in variety ‘UNR-01’.

14. The potato tuber, plant, plantlet, plant part, or plant cell of claim 1, further comprising one or more transgenes, wherein said one or more transgenes is introduced by backcrossing or genetic transformation into potato variety ‘UNR-01’.

15. An F₁ hybrid potato seed of potato variety ‘UNR-01’ produced by crossing the potato tuber, plant, plantlet, plant part, or plant cell of claim 1 with a different potato plant.

16. An F₁ hybrid potato plant or plant part produced by growing the potato seed of claim 15, wherein the plant part comprises at least one somatic cell of the F1 hybrid potato plant of ‘UNR-01’.

17. The potato tuber, plant, plantlet, plant part, or plant cell of claim 1, further comprising a single locus conversion, wherein the single locus conversion is introduced by backcrossing or genetic transformation into potato variety ‘UNR-01’.

18. A method of producing potato seed, comprising:
crossing the potato plant of claim 1 with itself or a second potato plant; and
harvesting a resulting potato seed.

19. A potato seed produced by the method of claim 18.

20. A potato plant, or a part thereof, produced by growing the seed of claim 19.

21. An F₁ hybrid seed produced by the method of claim 18.

22. A method of producing a potato plant, comprising:
transforming a transgene conferring an additional trait into the potato plant of claim 1, thereby producing a potato plant comprising the added trait.

23. A potato plant produced by the method of claim 22.

24. A method of introducing an additional trait into potato variety ‘UNR-01’, comprising:
(a) crossing the potato plant of claim 1 with another potato plant comprising an additional trait to produce F₁ progeny plants;
(b) selecting F₁ progeny plants that have the additional trait to produce selected F₁ progeny plants;
(c) backcrossing the selected F₁ progeny plants with at least one plant of variety ‘UNR-01’ to produce backcross progeny plants;
(d) selecting backcross progeny plants that have the additional trait; and
(e) repeating steps (c) and (d) two or more times in succession to produce selected second or higher backcross progeny plants that comprise the additional trait and in some examples all of the physiological and morphological characteristics of potato variety ‘UNR-01’ when grown in the same environmental conditions.

25. A potato plant produced by the method of claim 24.

26. A method of producing a progeny potato plant derived from potato variety ‘UNR-01’, wherein the method comprises applying plant breeding techniques to the potato plant of claim 1 to produce the progeny potato plant derived from potato variety ‘UNR-01’.

27. A method of producing a hybrid potato plant derived from potato variety ‘UNR-01’, comprising:
(a) preparing a progeny plant derived from potato variety ‘UNR-01’ by crossing the potato plant of claim 1 with a potato plant of a second variety;
(b) crossing the progeny plant with itself or a second plant to produce a seed of a progeny plant of a subsequent generation;
(c) growing a progeny plant of the subsequent generation from said seed and crossing the progeny plant of the subsequent generation with itself or a second plant; and
(d) repeating steps (b) and (c) for at least an additional generation with sufficient inbreeding to produce a hybrid potato plant derived from the potato variety ‘UNR-01’.

28. A hybrid plant produced by the method of claim 27.

29. A method of producing a commodity plant product comprising:
obtaining the potato tuber, plant, plantlet, plant part, or plant cell of claim 1 or a part thereof; and
producing the commodity plant product therefrom.

30. A food product made from the potato tuber of claim 1.

31. A heat-processed tuber product obtained from the potato tuber of claim 1.

32. A process for producing potato seed, comprising:
crossing the potato plant of claim 1 with a different potato plant; and
harvesting the seed.

33. An F1 potato seed produced by the process of claim 32.

34. An F1 potato plant produced by germinating the seed of claim 33.

35. A method of producing a genetic marker profile, comprising:
extracting nucleic acids from the seed of claim 33 or the plant germinated from said seed and genotyping said nucleic acids at one or more genetic loci, thereby producing a genetic marker profile.

36. A method of plant breeding, comprising
isolating nucleic acids from the seed of claim 33;
identifying one or more polymorphisms from the isolated nucleic acids; and
selecting a plant obtained from said seed having said one or more polymorphisms, wherein the plant is used in a plant breeding method.

37. A method of plant breeding, comprising:
isolating nucleic acids from the plant of claim 34;
identifying one or more polymorphisms from the isolated nucleic acids; and
selecting a plant having said one or more polymorphisms, wherein the plant is used in a plant breeding method.
